(19) United States
(12) Patent Application Publication
(10) Pub. No.: US 2025/0137054 A1
(43) Pub. Date: May 1, 2025

(54) COMPOSITION AND METHOD FOR EVALUATING RESPONSIVENESS OF EDARAVONE

(71) Applicant: MITSUBISHI TANABE PHARMA CORPORATION, Osaka (JP)

(72) Inventors: Aki KUSUNOKI, Osaka-shi (JP); Kinya OKADA, Osaka-shi (JP)

(73) Assignee: MITSUBISHI TANABE PHARMA CORPORATION, Osaka (JP)

(21) Appl. No.: 19/004,541

(22) Filed: Dec. 30, 2024

Related U.S. Application Data
(63) Continuation-in-part of application No. PCT/JP2023/024373, filed on Jun. 30, 2023.

(30) Foreign Application Priority Data
Jul. 1, 2022 (JP) 2022-106787

(51) Int. Cl. C12Q 1/6883; A61K 31/4152

(52) U.S. Cl. CPC C12Q 1/6883; A61K 31/4152; C12Q 2600/136; C12Q 2600/158

(57) ABSTRACT

A method for evaluating responsiveness of a target to edaravone includes administering a composition including edaravone to a target in need thereof such that the edaravone causes a change in expression level of a gene product in the target, and evaluating whether the target has responsiveness to edaravone based on the change in expression level of the gene product due to exposure of the target to the edaravone, The gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2.

### CROSS-REFERENCE TO RELATED APPLICATIONS

The present application is a continuation-in-part of and claims the benefit of priority to International Application No. PCT/JP2023/024373, filed Jun. 30, 2023, which is based upon and claims the benefit of priority to Japanese Application No. 2022-106787, filed Jul. 1, 2022. The entire contents of these applications are incorporated herein by reference.

### BACKGROUND OF THE INVENTION

### Field of the Invention

The present invention relates to a composition and a method for evaluating responsiveness of edaravone.

### Description of Background Art

Edaravone is an organic compound known as 5-methyl-2-phenyl-2,4-dihydro-3H-pyrazol-3-one (CAS number 89-25-8). Edaravone is used for medical purposes as a brain protective agent or as a therapeutic agent for amyotrophic lateral sclerosis (ALS), a type of neurodegenerative disease (Japanese Patent Publication No. H5-31523 and U.S. Pat. No. 6,933,310). The entire contents of these publications are incorporated herein by reference.

### SUMMARY OF THE INVENTION

According to one aspect of the present invention, a method for evaluating responsiveness of a target to edaravone includes administering a composition including edaravone to a target in need thereof such that the edaravone causes a change in expression level of a gene product in the target, and evaluating whether the target has responsiveness to edaravone based on the change in expression level of the gene product due to exposure of the target to the edaravone. The gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2.

According to another aspect of the present invention, a method for evaluating a target for a neurodegenerative disease includes administering a composition including edaravone to a target in need thereof, and evaluating whether the target has a neurodegenerative disease based on a change in expression level of a gene product in the target. The gene product is a gene product of one or more genes selected from KAZALD1, SBK1, UBE2L6, NTM, DCTD, ASF1B and FCSK.

According to yet another aspect of the present invention, a method for screening a substance for treating or preventing a neurodegenerative disease includes administering a composition including edaravone to a target in need thereof such that the edaravone causes a change in expression level of a gene product in the target, and selecting a test substance that treats or prevents a neurodegenerative disease based on the change in expression level of the gene product due to exposure of the target to the test substance. The gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2.

According to still another aspect of the present invention, a biomarker for diagnosing a neurodegenerative disease includes a gene product including one or more genes selected from KAZALD1, SBK1, UBE2L6, NTM, DCTD, ASF1B and FCSK.

According to still another aspect of the present invention, a biomarker for diagnosing edaravone responsiveness includes a gene product including one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows fluorescence microscope images showing cell morphology of neurons expressing wild-type and mutant TDP-43 proteins when exposed to ethacrynic acid (EA). The images in the lower row of FIG. 1 are enlarged views of the images in the upper row of FIG. 1;

FIG. 2 shows fluorescence microscope images showing cell morphology of neurons expressing wild-type and mutant TDP-43 proteins when exposed to ethacrynic acid and edaravone (The images in the lower row of FIG. 2 are enlarged views of the images in the upper row of FIG. 20);

FIG. 3 is a graph showing cell survival rates of neurons expressing wild-type and mutant TDP-43 proteins, depending on the presence or absence of exposure to ethacrynic acid and/or edaravone;

FIG. 4 is a diagram showing an overview of treatments for experimental groups used in gene product expression analysis;

FIG. 5 is a diagram showing an overview of procedures for gene product expression analysis;

FIG. 6 is a graph showing expression distribution of the experimental groups;

FIGS. 7A-7D are graphs showing expression changes of gene products depending on the presence or absence of edaravone and/or a pathological condition;

FIGS. 8A-8D are graphs showing expression changes of other gene products depending on the presence or absence of edaravone and/or a pathological condition;

FIGS. 9A-9D are graphs showing expression changes of yet other gene products depending on the presence or absence of edaravone and/or a pathological condition;

FIGS. 10A and 10B are graphs showing expression changes of yet other gene products depending on the presence or absence of edaravone and/or a pathological condition;

FIG. 11 is a graph showing expression changes of yet other gene products depending on the presence or absence of edaravone and/or a pathological condition;

FIG. 12 is a graph showing cell survival rates of neurons expressing wild-type and mutant TDP-43 proteins, and neurons not expressing TDP-43 proteins, depending on the presence or absence of exposure to edaravone;

FIG. 13 is a graph showing change rates in neurite length depending on the presence or absence of exposure to edaravone in neurons differentiated from iPS cells induced from cells derived from an ALS patient and a healthy individual;

FIG. 14 is a graph showing change rates in the number of death cells depending on the presence or absence of exposure to edaravone in neurons differentiated from iPS cells induced from cells derived from an ALS patient and a healthy individual;

FIG. 15 shows TDP-43 fluorescence intensities of fluorescence microscope images showing the intracellular localization of TDP-43 when exposed to edaravone in neurons differentiated from iPS cells induced from cells derived from an ALS patient and a healthy individual;

FIG. 16 shows motor neuron activity and response in healthy iPSC-derived motor neurons and ALS iPSC-derived motor neurons;

FIG. 17A is a representative graph of the network activity shown as a function of firing rate over time for the Healthy and ALS iPSC neurons before and 24 h after the edaravone treatment;

FIG. 17B is a normalized bar graph showing the difference in Firing rate and Bursting frequency for healthy and ALS motor neurons before and after the edaravone treatment at 24 and 48 h;

FIG. 18A is panels showing a representative raster plot where the y-axis is several randomized trials, and the x-axis is the time after stimulation; and

FIG. 18B shows normalized plots of the mean data points of the Pre/Post stimulus ratio as mean±SEM for both healthy and ALS motor neurons before and after the edaravone treatment at 24 and 48 h.

### DETAILED DESCRIPTION OF THE EMBODIMENTS

Embodiments will now be described with reference to the accompanying drawings, wherein like reference numerals designate corresponding or identical elements throughout the various drawings.

An embodiment of the present invention includes a composition containing edaravone. In one embodiment, the composition may be used as a pharmaceutical, or it may be used as a non-pharmaceutical, such as a reagent, for example.

In either case, by causing a change in expression level of a gene product of a specific gene in a target, an advantageous effect corresponding to a type or a degree of expression of the gene and its gene product can be obtained.

In the present specification, “edaravone” includes 5-methyl-2-phenyl-2,4-dihydro-3H-pyrazol-3-one itself and its tautomers, derivatives thereof, and salts, hydrates, or solvates thereof.

In one embodiment, edaravone preferably includes at least one selected from 5-methyl-2-phenyl-2,4-dihydro-3H-pyrazol-3-one and its tautomers, and salts, hydrates, or solvates thereof. That is, edaravone preferably includes at least one selected from isomers represented by the following structural formula (1) and structural formula (2), and salts, hydrates, or solvates thereof.

Edaravone can be produced, for example, using a production method described in European Patent Publication No. 208874.

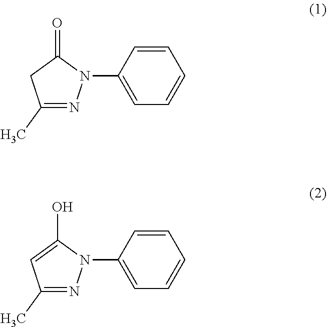

In the present specification, unless otherwise specified, the term “gene product” is used to include transcription products, reverse transcription products, and translation products derived from a gene, and can be one or more of these products.

A gene product may be one or more transcription products only, one or more reverse transcription products only, one or more translation products only, or a combination of one or more transcription products and one or more translation products.

Examples of transcription products include various types of RNA, such as mRNA and non-coding RNA. A transcription product is preferably mRNA, whether mature or not, and more preferably mature mRNA. An example of a reverse transcription product is cDNA. An example of a translation product is a protein produced through transcription and translation. A translation product is preferably a protein, regardless of the presence or absence of post-translational modification.

In one embodiment, the composition containing edaravone is preferably used as a pharmaceutical. That is, in one embodiment, the composition is a pharmaceutical composition containing edaravone as an active ingredient.

Using the above-described composition as a pharmaceutical, it is possible to change an expression level of a specific gene product in a target, for example, to maintain a brain function at a normal level or improve the brain function, or to contribute to treatment or prevention of diseases such as neurodegenerative diseases, muscle diseases, vascular disorders, and various inflammatory diseases.

In the present specification, the term “target” is not particularly limited as long as an expression level of a gene product can be measured. Examples of “target” include humans and non-human animals, as well as samples derived from these animals, regardless of the presence or absence of a disease or environments such as in vivo, ex vivo, or in vitro.

The humans and non-human animals are preferably mammals. That is, a target of exposure to the composition is preferably a mammal or a sample derived from a mammal.

Examples of non-human animals include rodents such as rats, mice, and guinea pigs; and non-human mammals such as monkeys, pigs, dogs, and cats. Examples of samples include, but are not limited to, one or more of tissues, cells, and body fluids. Examples of tissues include brain (such as cerebrum and cerebellum), spinal cord, stomach, pancreas, kidneys, liver, adrenal glands, skin, muscles (such as skeletal and smooth muscles), lungs, intestines (such as large and small intestines), heart, blood vessels, and the like. Examples of cells include differentiated cells, progenitor cells, or stem cells that form tissues. Taking brain-derived cells as an example, examples of brain-derived samples include neuronal cells (neurons), glial cells, neural-related differentiated cells such as astrocytes, neural stem cells, and the like. Examples of body fluids include liquid components such as cerebrospinal fluid, blood, serum, plasma, saliva, urine, and sweat, or extracts thereof.

These samples may typically be collected from living or dead animals using known methods. As other forms of cells, various cultured cells can be used, such as primary cultured cells, immortalized cell lines, or pluripotent stem cells such as ES cells and iPS cells. Among these, iPS cells may be induced from cells derived from an ALS patient or cells with an ALS risk mutation as a heterozygous mutation in the TARDBP gene. Cells differentiated from the pluripotent stem cells described above, for example, neurons, are also included in the target in an embodiment of the present invention.

In one embodiment, the above-described composition is suitably used for treating or preventing one or more of neurodegenerative diseases, muscle diseases, vascular disorders, and inflammatory diseases.

Examples of neurodegenerative diseases include neurodegenerative diseases with motor function impairment and neurodegenerative diseases with cognitive function impairment. Examples of neurodegenerative diseases with motor function impairment include amyotrophic lateral sclerosis (ALS), spinal muscular atrophy (SMA), progressive bulbar palsy, primary lateral sclerosis (PLS), arthrogryposis multiplex congenita (AMC), and the like. Examples of neurodegenerative diseases with cognitive function impairment include Alzheimer's disease, frontotemporal lobar degeneration (FTD), and the like. Examples of muscle diseases include muscular dystrophy, and the like. Examples of vascular disorders include cerebral infarction, cerebral hemorrhage, and the like.

Examples of inflammatory diseases include systemic inflammatory diseases such as multiple sclerosis and systemic sclerosis, and localized inflammatory diseases such as stomatitis

Among these diseases, the above-described composition as a pharmaceutical composition is used preferably for treating or preventing neurodegenerative diseases, more preferably for treating or preventing neurodegenerative diseases accompanied by motor dysfunction, and even more preferably for treating or preventing amyotrophic lateral sclerosis (ALS).

In the present specification, “treatment” includes suppression of progression, improvement, alleviation, complete cure, and the like of a disease.

In the specification, “prevention” includes prevention of onset of a disease, prevention of recurrence of a disease, and the like.

In one embodiment, the above-described composition is also preferably used for treatment or prevention of a disease accompanied by the presence of TDP-43 mutant protein or abnormal intracellular localization of TDP-43. Examples of such diseases include, but are not limited to, amyotrophic lateral sclerosis (ALS) and the like. Examples of TDP-43 mutant protein include, but are not limited to, C-terminal fragments of wild-type TDP-43 protein (for example, amino acid residues 208-414 from the N-terminus of wild-type TDP-43 protein) and point mutations of wild-type TDP-43 protein (for example, G294A, G298S, A315T, and Q343R). Examples of wild-type TDP-43 protein include an amino acid sequence described in Accession number: NP_031401.1.

Regardless of a disease, by using the above-described composition, an expression level of a specific gene product can be changed by exposure to edaravone. By the expression level of the gene product or the change in the expression level, cellular dysfunction or cellular damage such as cell death can be effectively suppressed. As a result, the disease can be treated or prevented, or symptoms of the disease can be alleviated. As shown in examples to be described later, the above-described composition is advantageous in that it is capable of treating or preventing diseases that potentially involve TDP-43 mutant protein, such as ALS. When TDP-43 mutant protein is present, it is thought that TDP-43 protein aggregates form within cells, making cellular damage more likely to occur. Therefore, by using the above-described composition, cellular damage can be effectively suppressed.

An edaravone-containing composition according to an embodiment of the present invention is used for causing a change in expression level of a specific gene product. As shown in the examples to be described later, expression levels of gene products of specific genes change depending on the presence or absence of exposure to edaravone. Further, the expression levels of these gene products also can change in specific diseases. Further, an expression change in the presence of edaravone can contribute to suppression of cellular damage, and consequently to prediction, or treatment or prevention of a disease caused by cellular damage.

In the present specification, the term “change” means that an amount of a substance is increased or decreased compared to a measured value or reference value of a comparison target, and also includes, in addition to a magnitude of the amount or a ratio, appearance of the amount from an undetectable state to a detectable state, and disappearance of the amount from a detectable state to an undetectable state.

Whether or not an expression level has changed is determined as follows: when a measured value of an expression level of a gene product in a target of determination is larger or smaller than a measured value or reference value of the expression level of the gene product in a comparison target, it is determined that the expression level has changed. The change in expression level may be determined based on a magnitude of a numerical number of the measured value itself or based on a magnitude of a ratio calculated from the measured value. Further, the change in expression level may be determined based on a magnitude of an arithmetic mean value or a median value calculated from multiple measured values or ratios, or it may be determined that it has changed based on the presence of a statistically significant difference.

The change in expression level is evaluated by comparing expression levels of measurable molecules derived from the same gene between two experimental groups as comparison targets, on a condition that the two experimental groups are of the same animal species or derived from the same animal species.

Examples of molecules for which expression levels can be measured include, but are not limited to, RNA, which is a transcription product, cDNA, which is a reverse transcription product of the RNA, proteins, which are translation products, and the like.

When evaluating the change in expression level using a ratio of measured values, it may be determined that the expression level has changed when a ratio (R2/R1) of a measured value R2 of an expression level of a gene product in an experimental group as a target of determination to a measured value or reference value R1 as a reference is, for example, 1.01 times or more, for example, 1.10 times or more, for example, 1.30 times or more, for example, 1.50 times or more, for example, 1.70 times or more, or for example, 2.0 times or more (log 2 ratio of 1 or more). In this case, it is also possible to determine that the experimental group as a target of determination has preferably changed such that the expression level is increased.

Further, it may be determined that the expression level has changed when the R2/R1 ratio is, for example, 0.99 times or less, for example, 0.90 times or less, for example, 0.80 times or less, for example, 0.70 times or less, for example, 0.60 times or less, or for example, 0.50 times or less (log 2 ratio of −1 or less). In this case, it is also possible to determine that the experimental group as a target of determination has preferably changed such that the expression level is decreased.

A composition according to an embodiment of the present invention is suitably used for causing a change in expression level of a transcription product of, or a translation product encoded by, one or more genes selected from a group of specific genes discovered through studies. The transcription product and translation product of which an expression level is changed may be one of these, or two or more of these.

In the following, names of genes of which expression levels of gene products change due to the presence of edaravone and/or a disease are exemplified but are not limited to these. The following genes are denoted as human genes unless otherwise specified. However, an embodiment of the present invention also includes orthologs of animal species other than humans. Base sequences or protein amino acid sequences of these genes can be searched for using a public database or the like.

In the present specification, gene groups of which expression levels of gene products change due to the presence of edaravone and/or a disease (the following (A) and (B)) are collectively referred to as “specific genes.” Description regarding specific genes applies to all embodiments described in the present specification unless otherwise specified.

### Specific Genes

Examples of genes of which expression levels of gene products increase due to the presence of a disease and the absence of edaravone, and decrease due to the presence of both a disease and edaravone, are shown below as (A).

In one embodiment, the above-described composition is used for decreasing an expression level of a gene product in a target with a disease, for any single gene, any combination of two or more genes, or a combination of all genes from the gene group (A).

In one embodiment, the above-described disease is preferably a neurodegenerative disease.

### Gene Group (A)

KAZALD1 (Kazal Type Serine Peptidase Inhibitor Domain 1)
SBK1 (SH3-binding domain kinase 1)
SCN2A (sodium voltage-gated channel alpha subunit 2)
UBE2L6 (ubiquitin conjugating enzyme E2 L6)
ALPL (alkaline phosphatase)
NTM (neurotrimin)
PTTG1 (regulator of sister chromatid separation; Securin)
ITGB4 (integrin subunit beta 4)
HAUS4 (HAUS augmin like complex subunit 4)
DCTD (dCMP deaminase)
MT2A (metallothionein 2A)
ASF1B (anti-silencing function 1B histone chaperone)
FCSK (fucose kinase)
MAST1 (microtubule associated serine/threonine kinase 1)

Further, examples of genes of which expression levels of gene products decrease due to the presence of a disease and the absence of edaravone and increase due to the presence of both a disease and edaravone, are shown below as (B).

In one embodiment, the above-described composition is used for increasing an expression level of a gene product in a target with a disease, for the gene group (B). In one embodiment, the above-described disease is preferably a neurodegenerative disease.

### Gene Group (B)

### FAIM2 (Fas Apoptotic Inhibitory Molecule 2)

KAZALD1 is a gene that encodes a type of protein belonging to an insulin growth factor (IGF) binding protein superfamily. An expression change of KAZALD1 can contribute to cell proliferation or a change in function or expression level of IGF. It has been found that an expression level of a gene product of this gene increases in the presence of TDP-43 mutant protein, which is a pathological condition of a neurodegenerative disease, and further, decreases in the presence of edaravone.

There is a possibility that an expression change of IGF is associated with a risk change of a neurodegenerative disease such as ALS through some mechanism. Therefore, it is thought that decreasing the expression level of KAZALD1 is useful in decreasing the risk of developing or progressing a neurodegenerative disease through regulation of IGF activity and enhancing the possibility of treatment or prognosis prediction of a neurodegenerative disease such as ALS.

SBK1 is a gene that encodes a type of serine/threonine protein kinase. An expression change of SBK1 can contribute to cancer progression or cell proliferation. It has been found that an expression level of a gene product of this gene increases in the presence of TDP-43 mutant protein, which is a pathological condition of a neurodegenerative disease, and further, decreases in the presence of edaravone.

It is thought that decreasing the expression level of SBK1 is useful in suppressing the occurrence of cellular damage by suppressing the formation of TDP-43 protein aggregates through phosphorylation or enhancing the possibility of treatment or prognosis prediction for a neurodegenerative disease such as ALS.

SCN2A is a gene that encodes a type of protein belonging to a voltage-gated sodium channel family in excitatory neurons. An expression change of SCN2A can contribute to occurrence of epilepsy or a developmental disorder. Further, during neuronal cellular damage, the function of SCN2A can become excessively activated, potentially worsening the cellular damage. It has been found that an expression level of a gene product of this gene increases in the presence of TDP-43 mutant protein, which is a pathological condition of a neurodegenerative disease, and further, decreases in the presence of edaravone.

By decreasing the expression level of SCN2A, the occurrence of cellular damage can be suppressed. Further, it is thought that it is useful in that, along with the decrease in the expression level of SCN2A, a possibility of performing treatment or prognosis prediction of a neurodegenerative disease such as ALS can be enhanced. In particular, it is thought that decreasing the expression level of SCN2A is advantageous in that the function of SCN2A can be suppressed during occurrence of neuronal cellular damage and as a result, progression of the cellular damage can be effectively suppressed.

UBE2L6 is a gene that encodes a type of protein belonging to a ubiquitin-conjugating enzyme family. It is thought that an expression change of UBE2L6 contributes to protein degradation and suppresses autophagy in a pathological condition such as cancer. It has been found that an expression level of a gene product of this gene increases in the presence of TDP-43 mutant protein, which is a pathological condition of a neurodegenerative disease, and further, decreases in the presence of edaravone.

When autophagy is suppressed, degradation of abnormal proteins such as TDP-43 protein aggregates may be hindered, potentially leading to progression of the pathological condition. Therefore, by decreasing the expression level of UBE2L6, autophagy can be promoted and degradation of abnormal proteins can be promoted. As a result, it is thought that it is useful in suppressing the occurrence of cellular damage or enhancing the possibility of treatment or prognosis prediction for a neurodegenerative disease such as ALS.

ALPL is a gene that encodes a type of protein belonging to an alkaline phosphatase family. ALPL is highly expressed in axons or dendrites in neurons. It has been found that an expression level of a gene product of this gene increases in the presence of TDP-43 mutant protein, which is a pathological condition of a neurodegenerative disease, and further, decreases in the presence of edaravone.

An increase in expression level of ALPL can contribute to occurrence or progression of a neurodegenerative disease such as ALS through some mechanism in neuronal cells. Therefore, it is thought that decreasing the expression level of ALPL is useful in suppressing the occurrence of cellular damage or enhancing the possibility of treatment or prognosis prediction for a neurodegenerative disease such as ALS.

NTM is a gene that encodes a type of protein belonging to an IgLON family of cell adhesion molecules. An expression change of NTM can contribute to elongation of neurites and axons, or cell-to-cell adhesion. It has been found that an expression level of a gene product of this gene increases in the presence of TDP-43 mutant protein, which is a pathological condition of a neurodegenerative disease, and further, decreases in the presence of edaravone. In an autoimmune neurological disease involving antibodies against the IgLON family, phosphorylated tau protein may accumulate in the thalamus and brainstem tegmentum, potentially leading to symptoms similar to those of a bulbar palsy-type motor neuron disease. Therefore, it is thought that decreasing the expression level of NTM is useful in reducing accumulation of abnormal proteins or adverse effects associated with the accumulation, thereby enhancing the possibility of treatment or prognosis prediction of various neurodegenerative diseases such as bulbar palsy-type ALS.

PTTG1 is a gene that encodes securin, which is a protein that contributes to cellular mitosis. An expression change of PTTG1 can contribute to regulation of cell division or tumor formation. It has been found that an expression level of a gene product of this gene increases in the presence of TDP-43 mutant protein, which is a pathological condition of a neurodegenerative disease, and further, decreases in the presence of edaravone.

An increase in expression level of PTTG1 can contribute to occurrence or progression of a neurodegenerative disease such as ALS through some mechanism related to cell division. Therefore, it is thought that decreasing the expression level of PTTG1 is useful in suppressing the occurrence of cellular damage or enhancing the possibility of treatment or prognosis prediction for a neurodegenerative disease such as ALS.

ITGB4 is a gene that encodes a type of protein belonging to an integrin family. An expression change of ITGB4 can contribute to cell proliferation, adhesion, and migration, or tissue formation and repair. It has been found that an expression level of a gene product of this gene increases in the presence of TDP-43 mutant protein, which is a pathological condition of a neurodegenerative disease, and further, decreases in the presence of edaravone.

An increase in expression level of ITGB4 can contribute to occurrence or progression of a neurodegenerative disease such as ALS through some mechanism related to cell proliferation or repair. Therefore, it is thought that decreasing the expression level of ITGB4 is useful in suppressing the occurrence of cellular damage or enhancing the possibility of treatment or prognosis prediction for a neurodegenerative disease such as ALS.

HAUS4 is a gene that encodes a type of protein that contributes to microtubule formation during cellular mitosis. An expression change of HAUS4 can contribute to regulation of cell division or elongation of axons and dendrites. It has been found that an expression level of a gene product of this gene increases in the presence of TDP-43 mutant protein, which is a pathological condition of a neurodegenerative disease, and further, decreases in the presence of edaravone.

An increase in expression level of HAUS4 has been confirmed in an ALS disease animal model and can contribute to occurrence or progression of a neurodegenerative disease such as ALS. Therefore, it is thought that decreasing the expression level of HAUS4 is useful in contributing to the formation or functional maintenance of normal neuronal cells, suppressing the occurrence of cellular damage, or enhancing the possibility of treatment or prognosis prediction for a neurodegenerative disease such as ALS.

DCTD is a gene that encodes a protein (dCMP deaminase) that contributes to deamination of dCMP to dUMP. An expression change of DCTD can contribute to regulation of nucleic acid biosynthesis. It has been found that an expression level of a gene product of this gene increases in the presence of TDP-43 mutant protein, which is a pathological condition of a neurodegenerative disease, and further, decreases in the presence of edaravone.

The upregulation of DCTD can lead to an imbalance of substrates or products in nucleic acid biosynthesis, potentially making DNA mutations more likely to occur during DNA replication. Therefore, it is thought that decreasing the expression level of DCTD is useful in contributing to the formation or functional maintenance of normal neuronal cells, suppressing the occurrence of cellular damage, or enhancing the possibility of treatment or prognosis prediction for a neurodegenerative disease such as ALS.

MT2A is a gene that encodes a type of protein that belongs to a metallothionein family. An expression change of MT2A can contribute to heavy metal metabolism, such as intracellular heavy metal concentration, or apoptosis. It has been found that an expression level of a gene product of this gene increases in the presence of TDP-43 mutant protein, which is a pathological condition of a neurodegenerative disease, and further, decreases in the presence of edaravone.

The upregulation of MT2A is thought to be caused in part by an oxidative stress. Therefore, it is thought that decreasing the expression level of MT2A is useful in contributing to the normal functional maintenance of neuronal cells by adjusting response to an oxidative stress, suppressing the occurrence of cellular damage, or enhancing the possibility of treatment or prognosis prediction for a neurodegenerative disease such as ALS.

ASF1B is a gene that encodes a type of protein belonging to a histone chaperone H3/H4 family. An expression change of ASF1B can contribute to regulation of cell division, proliferation, and aging. It has been found that an expression level of a gene product of this gene increases in the presence of TDP-43 mutant protein, which is a pathological condition of a neurodegenerative disease, and further, decreases in the presence of edaravone.

An increase in expression level of ASF1B can contribute to occurrence or progression of a neurodegenerative disease such as ALS through some mechanism related to cell division or proliferation. Therefore, it is thought that decreasing the expression level of ASF1B is useful in suppressing the occurrence of cellular damage or enhancing the possibility of treatment or prognosis prediction for a neurodegenerative disease such as ALS.

FCSK is a gene that encodes a type of protein belonging to a phosphotransferase family and using alcohol as a receptor. An expression change of FCSK can contribute to metabolism of fructose and mannose. It has been found that an expression level of a gene product of this gene increases in the presence of TDP-43 mutant protein, which is a pathological condition of a neurodegenerative disease, and further, decreases in the presence of edaravone.

An increase in expression level of FCSK can contribute to occurrence or progression of a neurodegenerative disease such as ALS through some mechanism related to intracellular glucose metabolism. Therefore, it is thought that decreasing the expression level of FCSK is useful in suppressing the occurrence of cellular damage or enhancing the possibility of treatment or prognosis prediction for a neurodegenerative disease such as ALS.

MAST1 is a gene that encodes a type of serine/threonine protein kinase. An expression change of MAST1 is mainly related to brain development and can contribute to expression at postsynapses or synaptic terminals of neuromuscular junctions. It has been found that an expression level of a gene product of this gene increases in the presence of TDP-43 mutant protein, which is a pathological condition of a neurodegenerative disease, and further, decreases in the presence of edaravone.

It is thought that decreasing the expression level of MAST1 is useful in suppressing the occurrence of cellular damage by suppressing the formation of TDP-43 protein aggregates through phosphorylation or enhancing the possibility of treatment or prognosis prediction for a neurodegenerative disease such as ALS.

FAIM2 is a gene that encodes a type of anti-apoptotic protein that protects cells from Fas-induced apoptosis. This gene is also known as protein lifeguard 2-like (LOC102551901) as a rat orthologue. An expression change of FAIM2 can contribute to a size of the cerebellum or a thickness of the internal granular layer, and development of Purkinje cells. It has been found that an expression level of a gene product of this gene decreases in the presence of TDP-43 mutant protein, which is a pathological condition of a neurodegenerative disease, and further, increases in the presence of edaravone.

Since apoptosis can be induced during neuronal damage, it is thought that the expression level of FAIM2 is decreased. Therefore, it is thought that increasing the expression level of FAIM2 is useful in enhancing the possibility of treatment or prognosis prediction for a neurodegenerative disease such as ALS by maintaining the survival of neuronal cells.

Further, FAIM2 is associated with the regulation of autophagy. Therefore, increasing the expression level of FAIM2 can suppress the formation of mutant SOD1 (Superoxide dismutase 1) protein aggregates, which is a pathological condition of a neurodegenerative disease such as ALS, or promote the degradation of aggregates of abnormal proteins such as TDP-43 and SOD1. As a result, it is thought that it is useful in enhancing the possibility of treatment or prognosis prediction for a neurodegenerative disease such as ALS.

An exposure method of the above-described composition is not particularly limited. Examples of the exposure method include administration to a living body, contact with a sample, and the like.

In the case of in vivo administration to a human or a non-human animal, the examples include oral administration and parenteral administration. In this case, the edaravone-containing composition is preferably a pharmaceutical composition containing edaravone. Examples of parenteral administration include intravenous, intramuscular, intraperitoneal, subcutaneous or intradermal injection, injection into the digestive tract, inhalation, and the like. These administrations may be a single rapid administration or multiple rapid administrations or may be a continuous administration such as infusion.

Contact with a sample can be performed, for example, using a liquid in which the above-described composition is dissolved or dispersed.

The above-described composition can be in a solid or liquid state at 1 atmosphere and 20° C., depending on an intended usage. Examples of dosage forms when the composition is in a solid state include tablets, capsules, powders, fine granules, granules, suppositories, and the like. Examples of dosage forms when the composition is in a liquid state include liquid agents, syrups, suspensions, injectables, infusions, and the like.

A liquid can be a solution containing a solvent or a dispersion containing a dispersing agent. The above-described composition may further contain additives as needed. These additives are preferably pharmaceutically acceptable.

In one embodiment, when an additive is contained in the composition, the additive can constitute a remainder of the composition either as a single type of additive alone or as a combination of two or more types of additives.

As the additives, those used in the present technical field can be used without particular restrictions. As the additives, for example, excipients, disintegrants or disintegration aids, binders, lubricants, coating agents, colorants, diluents, vehicles, solvents or solubilizing agents, isotonic agents, pH adjusters, stabilizers, propellants, adhesives, and the like can be used. These additives can each be used independently, or two or more of the additives can be used in combination.

As vehicles or diluents, for example, water, electrolyte-containing water such as saline, monovalent alcohols with 1 to 3 carbon atoms such as methanol, ethanol, and propanol, or aqueous liquids used for cell culture can be used. These aqueous liquids can each be used independently or two or more of the aqueous liquids can be used in combination, as solvents or dispersing agents.

The content of edaravone in the composition can be appropriately changed depending on an applicable target. However, taking as an example the case of administration to a living mammal, regardless of the form of administration, the content of edaravone can be set to an amount that can be administered in a range of preferably 0.01 μg/kg body weight or more and 10 mg/kg body weight or less per day.

In one embodiment, taking as an example the case of administration to an adult human, regardless of the form of administration, the content of edaravone in the composition can be set to an amount that can be administered per day in a range of preferably 10 mg or more and 150 mg or less, more preferably 20 mg or more and 120 mg or less, and even more preferably 90 mg or more and 120 mg or less. The above content can also be set as, for example, a therapeutically effective amount.

When exposing samples such as tissues or cells collected from animals or in vitro samples, the content of edaravone in the composition, taking a liquid form as an example, is preferably 0.1 μmol/L or more and 1000 μmol/L or less, more preferably 0.5 μmol/L or more and 500 μmol/L or less, even more preferably 0.75 μmol/L or more and 300 μmol/L or less, still more preferably 10 μmol/L or more and 300 μmol/L or less, and most preferably 50 μmol/L or more and 250 μmol/L or less.

In any form, when the content of edaravone is in the above-described ranges, occurrence of an adverse effect such as cellular damage, or progression of a disease to a target can be effectively suppressed.

In one embodiment, whether or not there is a possibility that a target has a disease can be evaluated by using an expression level of a gene product of a specific gene described above or a change in the expression level as an indicator. That is, the present embodiment relates to a method for evaluating a possibility of having a disease. By adopting this evaluation method, early detection of a disease or assessment of the need for appropriate treatment can be facilitated, potentially contributing to the improvement of a patient's quality of life.

In one embodiment, a disease that can be evaluated using the present method is preferably a neurodegenerative disease, more preferably ALS, and even more preferably ALS associated with the presence of TDP-43 mutant protein or abnormal intracellular localization of TDP-43.

In the present specification, when describing an embodiment of a method, unless otherwise specified, the method may include performing only one process or may include performing multiple processes at the same time or in any order. Further, in a case of an embodiment that can include multiple processes, unless otherwise specified, one or more other processes may be further interposed between two processes, two processes may be performed consecutively such that no other processes are interposed therebetween, or two or more processes may be performed at the same time. The processes according to the embodiments described in the present specification may be appropriately combined.

Specifically, the present method includes a process of evaluating whether or not there is a possibility that a target has a disease based on an expression level, or a changed in the expression level, of a gene product of one or more genes among specific genes in the target.

An applicable target of the present method is preferably a mammal or a sample derived from the mammal. More specifically, an applicable target of the present method is preferably a brain of a mammal or a sample derived from a brain.

Further, as an applicable target of the present method, a sample collected from a living or dead mammal can be preferably used, and the mammal is more preferably a human or a non-human animal.

In one embodiment, the present method can also be performed as a method to assist in detecting a disease from a target, and the disease is preferably a neurodegenerative disease. In one embodiment, the present method can also be performed under an in vitro condition.

In one embodiment, the present method includes a process A1 of measuring an expression level of a gene product of a specific gene in a target. That is, in the present process A1, an expression level of a transcription product of, or a translation product encoded by, one or more specific genes selected from KAZALD1, SBK1, SCN2A, UBE2L6, ALPL, NTM, PTTG1, ITGB4, HAUS4, DCTD, MT2A, ASF1B, FCSK, MAST1 and FAIM2 is measured. In the present method, one or more types of gene products of specific genes can be used as indicators of a possibility of having diseases such as neurodegenerative diseases or as biomarkers for evaluation. The measurement of an expression level may be performed in vitro or in vivo.

When quantifying a transcription product as a target as an expression level of a gene product, for example, RNA can be extracted from a sample as a measurement target using a conventional method, and, when necessary, after obtaining cDNA, which is a reverse transcription product, measurement may be performed using methods known in the present technical field, such as Northern blot, RT-PCR, nucleic acid array, and RNA-seq. Further, when necessary, correction of a measured value can be performed based on expression levels of housekeeping genes such as GAPDH and β-actin.

When quantifying a translation product (that is, protein) as a target as an expression level of a gene product, measurement may be performed using immunological methods known in the present technical field, such as ELISA, flow cytometry, Western blot method, or immunohistochemical staining, for example, using a sample or its solution as a measurement target. When necessary, correction of a measured value can be performed based on the protein expression levels of the housekeeping genes described above.

As other methods for quantifying a translation product, mass spectrometry, high-performance liquid chromatography, gas chromatography, NMR analysis, or a combination of these methods can be used. Examples of additional methods for measuring a translation product as a target include measuring the activity of the translation product and considering the measured activity as an expression level or administering in vivo a ligand that specifically binds to the translation product.

In one embodiment, the present method includes, in addition to the process A1, a process B1 of evaluating whether or not there is a possibility that a target has a disease based on a measured expression level of a gene product or a change in the expression level.

In order to evaluate the possibility of a disease in the process B1, for example, a method including at least one of the following processes B11, B12 and B13 can be adopted.

In one embodiment, the present method can include, for example, performing at least one of the processes B12 and B13 after the process A1.

In one embodiment, the present method can include, for example, performing the process B11 before or after the process A1 or simultaneously with the process A1, and then performing the processes B12 and B13 in this order.

The process B11 is a process of using a normal target, that is, a healthy animal not affected by a disease or a sample derived from such an animal, as a control group, to measure an expression level of a gene product in the sample. The expression level of the gene product in the control group can also be used as a reference value.

The process B12 is a process of comparing the expression level of the gene product in the normal target (control group) with an expression level of a gene product in an evaluation target.

The process B13 is a process of comparing the normal target (control group) with the evaluation target and evaluating that the evaluation target has a high possibility of having a disease when a change in expression level of a gene product of one or more genes among specific genes is confirmed.

The evaluation of the change in expression level is preferably performed based on the presence or absence of a statistically significant difference from a point of view of improving the evaluation accuracy.

The comparison of expression levels between experimental groups can be performed by comparing transcription products derived from the same gene or translation products derived from the same gene, under the same or similar experimental conditions.

Specifically, as shown in the examples to be described later, an expression level of a gene product of one or more genes belonging to the gene group (A) among the specific genes described above can increase compared to the normal target due to the presence of a disease (for example, a neurodegenerative disease). Therefore, when an expression level of a gene product of one or more genes belonging to the gene groups (A) is increased compared to the expression level in the normal target, it can be evaluated that the evaluation target or an animal from which the target is obtained has a high possibility of having a disease (for example, a neurodegenerative disease).

On the other hand, when an expression level of a gene product of one or more genes belonging to the gene group (A) among the specific genes described above is decreased or remains the same compared to the expression level in the normal target, it can be evaluated that the evaluation target or an animal from which the target is obtained has a low possibility of having a disease (for example, a neurodegenerative disease).

From a point of view of improving the evaluation accuracy regarding the possibility of having a disease and making it easier to exclude the possibility of a disease other than the one being evaluated, it is preferable to performed the evaluation by comparing expression levels of gene products with two or more genes among the specific genes as targets, and it is more preferable to perform the evaluation by comparing expression levels of gene products with all the specific genes described above as targets.

Further, an expression level of a gene product of genes belonging to the gene group (B) among the specific genes described above can decrease compared to the normal target due to the presence of a disease. Therefore, when an expression level of a gene product of the genes belonging to the gene groups (B) is decreased compared to the expression level in the normal target, it can be evaluated that the evaluation target or an animal from which the target is obtained has a high possibility of having a disease (for example, a neurodegenerative disease).

On the other hand, when an expression level of a gene product of the gene group (B) is increased or remains the same compared to the expression level in the normal target, it can be evaluated that the evaluation target or an animal from which the target is obtained has a low possibility of having a disease (for example, a neurodegenerative disease).

From a point of view of further improving the evaluation accuracy, it is preferable to perform the evaluation by comparing expression levels of gene products with all the genes belonging to the gene groups (A) and (B) as targets.

Alternatively or additionally, the possibility of the presence or absence of a disease can also be evaluated based on a cutoff value of an expression level of a gene product by setting the cutoff value as a reference value or a threshold. For example, when an expression level of a gene product of one or more genes among the specific genes described above is increased compared to the cutoff value (the gene group (A)) or decreased compared to the cutoff value (the gene group (B)), it can be evaluated that the evaluation target or an animal from which the target is derived has a high possibility of having a disease (for example, a neurodegenerative disease).

The “cutoff value” can be set, for example, as an expression level that satisfies, at a predetermined level, at least one of sensitivity and specificity when the presence or absence or severity of a disease is evaluated based on the cutoff value. Further, an expression level of a gene product in a healthy animal or a healthy human can also be used as the cutoff value. The cutoff value can be appropriately set according to the type of disease or the degree of strictness of the evaluation. Further, in order to evaluate the severity of a disease, multiple cut-off values may be set to stratify the possibility of having a disease.

In one embodiment, whether or not there is a possibility that a target has responsiveness to edaravone can be evaluated by using an expression level of a gene product of a specific gene described above or a change in the expression level as an indicator. That is, the present embodiment relates to a method for evaluating responsiveness to edaravone. This method allows for earlier and simpler evaluation and prediction of the effects of edaravone on a disease.

An applicable target of the present method is preferably a target that has or is suspected of having a neurodegenerative disease. The applicable target is more preferably a patient with a neurodegenerative disease or a mammal that serves as a disease model, or a sample derived from such a mammal. In this case, the mammal is more preferably a human or a non-human animal. More specifically, the applicable target of the present evaluation method is preferably a sample collected from a patient, or a brain of a mammal or a sample derived from a brain.

Further, the disease as an applicable target of the present method is preferably a neurodegenerative disease, more preferably ALS, and even more preferably ALS accompanied by the presence of TDP-43 mutant protein or abnormal intracellular localization of TDP-43.

Specifically, the present method preferably uses a target that has or is suspected of having a disease and has been exposed to edaravone. The method includes a process of evaluating whether or not there is a possibility that a target has responsiveness to edaravone based on a change in expression level of a gene product of one or more genes among specific genes in the target.

In one embodiment, the present method can also be used as a method to assist in predicting whether or not a target (for example, a target that has or is suspected of having a neurodegenerative disease) will effectively respond to treatment with edaravone.

In one embodiment, the present method can also be performed under an in vitro condition.

In the following, in the present embodiment, an experimental group to which a target that has or is suspected of having a disease and has been exposed to edaravone belongs is also referred to as an “exposed disease group.”

In one embodiment, the present method includes a process A2 of measuring an expression level of a gene product of a specific gene in a target (exposed disease group) that has or is suspected of having a disease and has been exposed to edaravone. That is, in the present process, an expression level of a transcription product of, or a translation product encoded by, one or more specific genes selected from KAZALD1, SBK1, SCN2A, UBE2L6, ALPL, NTM, PTTG1, ITGB4, HAUS4, DCTD, MT2A, ASF1B, FCSK, MAST1, and FAIM2 in a target that has or is suspected of having a disease and has been exposed to edaravone is measured.

The measurement of the expression level of the transcription product or the translation product may be performed using the same measurement methods as in the embodiments described above.

Whether or not a target used in the present method has or is suspected of having a disease can be evaluated using, for example, the method in the embodiments described above. Additionally or alternatively, when performing evaluation regarding a neurodegenerative disease as one form of disease, it is also possible to evaluate that a target has or is suspected of having a neurodegenerative disease based on the presence of TDP-43 mutant protein or based on abnormal intracellular localization of TDP-43. The presence of TDP-43 mutant protein or the abnormal intracellular localization of TDP-43 can be determined, for example, by subjecting a sample such as tissue or cells to an immunological method described above.

Further, for example, animals including humans that have been evaluated as having or suspected of having neurodegenerative diseases by performing examinations such as electromyography on their living bodies, or samples from such animals, can be used.

Exposure of a target to edaravone may be performed, for example, by in vivo administration, or after collecting a sample from an animal that has not been exposed to edaravone, the sample may be brought into contact with edaravone under an in vitro condition.

In the case where edaravone has been administered in vivo, for example, a sample collected from a living body such as a human patient or an animal after the administration may be subjected to expression level measurement. In this case, the sample is treated as target that has been exposed to edaravone.

In either case, the exposure to edaravone may use edaravone itself, or may use a composition according to the embodiments described above.

An exposure amount of a target to edaravone can be appropriately changed depending on a purpose. For example, in the case of in vivo administration, the exposure amount of edaravone can be set to a range of preferably 0.01 μg/kg body weight or more and 10 mg/kg body weight or less per day.

In one embodiment, taking as an example the case of administration to an adult human, regardless of the form of administration, the exposure amount of edaravone can be set to an amount that can be administered per day in a range of preferably 10 mg or more and 150 mg or less, more preferably 20 mg or more and 120 mg or less, and even more preferably 90 mg or more and 120 mg or less.

For the in vivo administration, for example, the oral and/or parenteral administration methods described above can be adopted.

When exposing samples such as tissues or cells collected from animals or in vitro samples, an exposure concentration of edaravone is preferably 0.1 μmol/L or more and 1000 μmol/L or less, more preferably 0.5 μmol/L or more and 500 μmol/L or less, even more preferably 0.75 μmol/L or more and 300 μmol/L or less, still more preferably 10 μmol/L or more and 300 μmol/L or less, and most preferably 50 μmol/L or more and 250 μmol/L or less.

An exposure time of a sample to edaravone can be appropriately changed depending on a purpose. For example, the exposure time can be preferably 1 hour or more and 120 hours or less, and more preferably 12 hours or more and 60 hours or less, provided that the concentration is as described above.

In the present method, from a point of view of further improving the evaluation accuracy, the target may be further exposed to a stress inducer simultaneously with, or before or after, the exposure to edaravone.

Examples of stress inducers include compounds that cause a stress in an exposure target, making it more susceptible to cellular damage. Examples of such compounds include one or more of endoplasmic reticulum stress agents, osmotic stress agents, oxidative stress agents, and the like. These are preferably used in an in vitro environment.

Examples of endoplasmic reticulum stress agents include thapsigargin, tunicamycin, dithiothreitol, and the like.

Examples of osmotic stress agents include sorbitol, sodium chloride, and the like.

Examples of oxidative stress agents include ethacrynic acid, arsenite such as sodium arsenite, quinone compounds such as tert-butylhydroquinone, and the like.

When stress inducers are included, the contents of stress inducers can be appropriately changed depending on the types and combination of the compounds, but can each be independently set to, for example, 0.1 μmol/L or more and 1000 μmol/L or less, for example, 1 μmol/L or more and 100 μmol/L or less.

An exposure time of a stress inducer can be in the same range as the exposure time of edaravone.

In one embodiment, the present method includes, in addition to the process A2, a process B2 of evaluating whether or not there is a possibility that a target has responsiveness to edaravone based on a measured change in expression level of a gene product.

In order to evaluate the possibility of responsiveness to edaravone in the process B2, for example, a method including at least one of the following processes B21, B22 and B23 can be adopted.

In one embodiment, the present method can include, for example, performing at least one of the processes B22 and B23 after the process A2.

In one embodiment, the present method can include, for example, performing the process B21 before or after the process A2, or simultaneously with the process A2, and then performing the processes B22 and B23 in this order.

The process B21 is a process of measuring an expression level of a gene product in an unexposed disease group, using a target that has or is suspected of having a disease and has not been exposed to edaravone as the unexposed disease group. From a point of view of improving the evaluation accuracy, it is preferable that the target in the unexposed disease group and the target in the exposed disease group are the same except for the presence or absence of exposure to edaravone.

The process B22 is a process of comparing the expression level of the gene product in the target belonging to the unexposed disease group with the expression level of the gene product in the evaluation target (the exposed disease group).

The process B23 is a process of comparing the unexposed disease group with the exposed disease group, and evaluating that the target has a high possibility of having responsiveness to edaravone when a change in expression level of a gene product of one or more genes among specific genes is confirmed.

The evaluation of the change in expression level may be performed by setting an arbitrary threshold or may be performed based on a statistically significant difference. The evaluation of the change in expression level is preferably performed based on the presence or absence of a statistically significant difference from a point of view of improving the evaluation accuracy.

Specifically, an expression level of a gene product of one or more genes belonging to the gene group (A) among the specific genes described above can increase in the presence of a disease (for example, a neurodegenerative disease) compared to a normal target, and can further decrease by exposure to edaravone compared to the case where only a disease (for example, a neurodegenerative disease) is present. Therefore, for one or more genes in the gene group (A), when an expression level of a gene product in the exposed disease group is decreased compared to an expression level of a gene product in the unexposed disease group, it can be evaluated that a target that has or is suspected of having a disease (for example, a neurodegenerative disease) has a high possibility of having responsiveness to edaravone.

On the other hand, for the gene group (A), when an expression level of a gene product in the exposed disease group is increased or remains the same compared to an expression level of a gene product in the unexposed disease group, it can be evaluated that a target that has or is suspected of having a disease (for example, a neurodegenerative disease) has a low possibility of having responsiveness to edaravone.

From a point of view of improving the evaluation accuracy regarding the responsiveness to edaravone, it is preferable to performed the evaluation by comparing expression levels of gene products with multiple genes among the specific genes as targets, and it is more preferable to perform the evaluation by comparing expression levels of gene products with all the specific genes described above as targets.

Further, an expression level of a gene product of genes belonging to the gene group (B) among the specific genes described above can decrease in the presence of a disease (for example, a neurodegenerative disease) compared to a normal target, and can further increase by exposure to edaravone compared to the case where only a disease (for example, a neurodegenerative disease) is present. Therefore, for genes belonging to the gene group (B), when an expression level of a gene product in the exposed disease group is increased compared to an expression level of a gene product in the unexposed disease group, it can be evaluated that a target that has or is suspected of having a disease (for example, a neurodegenerative disease) has a high possibility of having responsiveness to edaravone.

On the other hand, for the gene group (B), when an expression level of a gene product in the exposed disease group is decreased or remains the same compared to an expression level of a gene product in the unexposed disease group, it can be evaluated that a target that has or is suspected of having a disease (for example, a neurodegenerative disease) has a low possibility of having responsiveness to edaravone.

From a point of view of further improving the evaluation accuracy, it is preferable to perform the evaluation by comparing expression levels of gene products with all the genes belonging to the gene groups (A) and (B) as targets.

From a point of view of more accurately evaluating the possibility of responsiveness to edaravone, it is also preferable to further use a normal target, that is, a healthy animal not affected by a disease or a sample derived from such an animal, as a control group and to perform comparison with an expression level of a gene product in the evaluation target. That is, in the present embodiment, it is preferable to perform the comparison using two or three experimental groups among the exposed disease group, the unexposed disease group, and the normal group.

An expression level of a gene product of the specific gene described above can be increased or decreased in the presence of a disease compared to a normal target, and further can be decreased or increased by exposure to edaravone. Based on such a useful expression profile, the present embodiment can evaluate the possibility of a therapeutic effect of edaravone administration or assist in effective prediction of therapeutic responsiveness.

Specifically, the process B2 in the present embodiment preferably includes at least one of the following processes B24, B25 and B26.

In one embodiment, the present method can include, for example, performing at least one of the processes B25 and B26 after the process A2.

In one embodiment, the present method can include, for example, performing the process B24 before or after the process A2, or simultaneously with the process A2, and then performing the processes B25 and B26 in this order.

The process B24 is a process of measuring an expression level of a gene product in a normal target (control group). The normal target is preferably not exposed to edaravone.

The process B25 is a process of comparing an expression level of a gene product in a target that has or is suspected of having a disease and has been exposed to edaravone (exposed disease group) with an expression level of a gene product in a normal target (control group).

The process B26 is a process of evaluating that there is a possibility that a target that has or is suspected of having a disease has responsiveness to edaravone and edaravone is highly effective on the target, when no change in an expression level of a gene product in a target in an exposed disease group is confirmed compared to an expression level of a gene product in a normal target for one or more specific genes. When evaluated in this way, for example, it can also be evaluated that there is a possibility that an exposure amount or exposure frequency of edaravone in an evaluation target is appropriate.

On the other hand, when a change in an expression level of a gene product in a target in an exposed disease group is confirmed compared to an expression level of a gene product in a normal target for one or more specific genes, it can be evaluated that there is a possibility that an evaluation target does not have responsiveness to edaravone, or it can be evaluated that there is a possibility that the responsiveness to edaravone is low and the effect of edaravone is low, or the responsiveness to edaravone is high and the effect of edaravone is very high. When evaluated in this way, depending on an increase or decrease in expression level or a degree of the increase or decrease, it can also be evaluated that, for example, there is a possibility that an exposure amount or exposure frequency of edaravone in an evaluation target is excessive or insufficient.

Further, taking the gene group (A) as an example, when an expression level of a gene product in the exposed disease group is lower than an expression level of a gene product in the unexposed disease group and higher than an expression level of a gene product in a normal target (control group), it can also be evaluated that there is a possibility that an evaluation target has low responsiveness to edaravone or there is a possibility that an exposure amount or exposure frequency of edaravone is insufficient.

Based on such evaluation, for example, a process of determining whether or not there is a need for appropriate treatment of a target may be further performed to allow monitoring of the effectiveness of edaravone treatment or assisting in treatment optimization. Examples of such a need include increasing or decreasing the exposure amount or exposure frequency of edaravone or implementing measures other than exposure to edaravone.

In any case, the evaluation of the change in expression level may be performed by setting an arbitrary threshold or may be performed based on a statistically significant difference. The evaluation of the change in expression level is preferably performed based on the presence or absence of a statistically significant difference from a point of view of improving the evaluation accuracy.

As another embodiment of a method for evaluating edaravone responsiveness, for example, the process B2 in the present embodiment preferably includes the processes B21 and B24, as well as the following processes B27 and B28.

In one embodiment, the present method can include, for example, performing the processes A1, B21 and B24 simultaneously or in any order, and then performing the processes B27 and B28 in this order.

The process B27 is a process of comparing an expression level of a gene product of a specific gene in a healthy individual (control group) with an expression level of a gene product of a specific gene in an unexposed disease group.

The process B28 is a process of evaluating that a target in the unexposed disease group or a patient or animal from which the target is sampled has a high possibility of responding to treatment with edaravone, when it is determined that there is a change in expression level between the two groups.

That is, the present embodiment is an example of a method for distinguishing the responsiveness to edaravone using, as a biomarker, an expression level of a gene product of a specific gene that has increased or decreased due to a disease compared to a healthy individual.

In one embodiment, the present method can also be used as a method for assisting in prediction of whether or not a target that has or is suspected of having a disease will effectively respond to treatment with edaravone.

When comparing the control group with the unexposed disease group, when an expression level of a gene product in the unexposed disease group is higher than an expression level of a gene product in the normal group for one or more genes in the gene group (A), it can be evaluated that a target that has or is suspected of having a disease (for example, a neurodegenerative disease) has a high possibility of having responsiveness to edaravone.

On the other hand, when comparing the control group with the unexposed disease group, when an expression level of a gene product in the unexposed disease group is lower than or equal to an expression level of a gene product in the normal group for the gene group (A), it can be evaluated that a target that has or is suspected of having a disease (for example, a neurodegenerative disease) has a low possibility of having responsiveness to edaravone.

Similarly, for genes belonging to the gene group (B), when comparing the control group with the unexposed disease group, when an expression level of a gene product in the unexposed disease group is lower than an expression level of a gene product in the normal group, it can be evaluated that a target that has or is suspected of having a disease (for example, a neurodegenerative disease) has a high possibility of having responsiveness to edaravone.

On the other hand, when comparing the control group with the unexposed disease group, when an expression level of a gene product in the unexposed disease group is higher than or equal to an expression level of a gene product in the normal group for the gene group (B), it can be evaluated that a target that has or is suspected of having a disease (for example, a neurodegenerative disease) has a low possibility of having responsiveness to edaravone.

In one embodiment, by using an expression level of a gene product of a specific gene described above or a change in the expression level as an indicator, any candidate substance can be selected as a substance capable of treating or preventing a neurodegenerative disease. That is, the present embodiment relates to a method for screening a substance capable of treating or preventing a neurodegenerative disease. With this method, it is possible to determine, using a method different from previous methods, whether an unknown substance is a substance capable of treating or preventing a neurodegenerative disease. As a result, it is possible to screen the substance as a drug candidate at an earlier stage, contributing to the early development of pharmaceuticals.

Specifically, the method for screening a substance capable of treating or preventing a neurodegenerative disease includes: a process A3 of exposing a target to a test substance; and a process B3 of selecting a test substance as a substance capable of treating or preventing a neurodegenerative disease based on a change in expression level of a gene product of a specific gene in a target exposed to the test substance.

In the present method, it is preferable that edaravone is excluded from the test substances. That is, it is preferable that a target used for substance evaluation in the present method is exposed to a test substance but is not exposed to edaravone.

In the present method, a target to be exposed to a test substance is preferably a mammal that has or is suspected of having a neurodegenerative disease, or a sample derived from a mammal that has or is suspected of having a neurodegenerative disease, and more preferably the mammal is a human. More specifically, an applicable target of the present method is preferably a brain of a mammal or a sample derived from a brain.

Further, the neurodegenerative disease as an evaluation target is preferably ALS, and more preferably ALS accompanied by the presence of TDP-43 mutant protein or abnormal intracellular localization of TDP-43.

The test substance is not particularly limited and examples thereof include organic low molecular weight compounds, nucleic acids, proteins, peptides, antibodies, natural compounds, and the like.

In the process A3, the method for exposing a target to a test substance is not particularly limited and may be performed in vitro or in vivo, for example, using a method similar to that in the embodiments described above.

When exposure of a target to a test substance is performed in vitro, for example, it can be performed by bring the target into contact with a liquid in which the test substance is dissolved or dispersed. Examples of the liquid include saline, buffer, or culture medium containing the test substance.

When exposure of a target to a test substance is performed in vivo, for example, the test substance itself or a liquid in which the test substance is dissolved or dispersed can be administered to a mammal. The administration method may be oral or parenteral.

In the process B3, an expression level of a gene product of a specific gene is measured, and based on a measured change in expression level, a test substance is determined to be a substance capable of treating or preventing a neurodegenerative disease.

In order to determine in the process B3 that a test substance is an intended substance, a method including at least one of the following processes B31, B32 and B33 can be adopted.

In one embodiment, the present method can include, for example, performing at least one of the processes B32 and B33 after the process A3.

In one embodiment, the present method can include, for example, performing the process B31 before or after the process A3, or simultaneously with the process A3, and then performing the processes B32 and B33 in this order.

The process B31 is a process of measuring an expression level of a gene product of a specific gene in an unexposed group, using a target that has not been exposed to a test substance and edaravone and has or is suspected of having a neurodegenerative disease as the unexposed group.

The process B32 is a process of comparing an expression level of a gene product in the unexposed group with an expression level of a gene product in a target that has been in contact with a test substance (test substance-exposed group).

The process B33 is a process of comparing the unexposed group and the exposed group, and when a change in expression level of the gene product is confirmed for one or more specific genes, determining that the test substance is a substance capable of treating or preventing a neurodegenerative disease and selecting the substance.

The determination of the change in expression level may be performed by setting an arbitrary threshold or may be performed based on a statistically significant difference. The determination of the change in expression level is preferably performed based on the presence or absence of a statistically significant difference from a point of view of improving the determination accuracy.

An expression level of a gene product of one or more genes belonging to the gene group (A) among the specific genes described above can increase due to the presence of a neurodegenerative disease. Then, a test substance with similar efficacy to edaravone can decrease an expression level of a gene product in a target. Therefore, when an expression level of a gene product of one or more genes among the specific genes in the test substance-exposed group is decreased compared to an expression level in the unexposed group, it can be determined that the test substance is a substance capable of treating or preventing a neurodegenerative disease.

On the other hand, when an expression level of a gene product of one or more genes belonging to the gene group (A) among the specific genes in the test substance-exposed group is increased or remains the same compared to an expression level in the unexposed group, it can be determined that the test substance is not a substance capable of treating or preventing a neurodegenerative disease or such a possibility is low.

An expression level of a gene product of genes belonging to the gene group (B) among the specific genes described above can decrease due to the presence of a neurodegenerative disease. Then, a test substance with similar efficacy to edaravone can increase an expression level of a gene product in a target. Therefore, when an expression level of a gene product of the genes in the test substance-exposed group is increased compared to an expression level in the unexposed group, it can be determined that the test substance is a substance capable of treating or preventing a neurodegenerative disease.

On the other hand, when an expression level of a gene product of the genes belonging to the gene group (B) in the test substance-exposed group is decreased or remains the same compared to an expression level in the unexposed group, it can be determined that the test substance is not a substance capable of treating or preventing a neurodegenerative disease or such a possibility is low.

From a point of view of improving the accuracy and reproducibility of the determination of the test substance and performing the screening more effectively, it is preferable to perform the determination by comparing expression levels of gene products of multiple genes among the specific genes, and it is more preferable to perform the determination by comparing all the specific genes described above.

From a point of view of more accurately determining a degree of contribution of the test substance to the treatment or prevention of a disease, the process B3 preferably includes a process B35 of further using a target that has not been exposed to the test substance but has been exposed to edaravone as an edaravone-exposed group, and comparing with an expression level of a gene product in the test substance-exposed group.

That is, it is preferable to compare expression levels in each target using three experimental groups including the test substance-exposed group, the edaravone-exposed group, and the unexposed group. That is, in the present embodiment, the unexposed group can be used as a negative control, and the edaravone-exposed group can be used as a positive control.

The targets used in these three experimental groups are preferably mammals that have or are suspected of having a neurodegenerative disease, or samples derived from mammals that have or are suspected of having a neurodegenerative disease. More specifically, applicable targets of the present determination method are preferably brains of mammals or samples derived from brains of mammals.

In this case, preparing groups with the same concentrations of the test substance and edaravone to be exposed is preferable in improving the accuracy and reproducibility of the evaluation.

In one embodiment of the present method, as the process B34, a process of measuring an expression level of a gene product of a specific gene in the edaravone-exposed group is performed. When the process B34 is performed, it may be performed before the process B35.

In one embodiment, the present method can include performing the processes A3, B31 and B34 simultaneously or in any order, and then performing (a) the processes B32 and B33 in this order, and/or (b) the process B35. When performing both the groups (a) and (b), at least the process B32 may be performed before the processes B33 and B35, and the processes B33 and B35 can be performed in any order or at the same time.

The process B35 is a process of comparing an expression level of a gene product in the test substance-exposed group with an expression level of a gene product in a target that has not been exposed to the test substance but has been exposed to edaravone.

Expression levels of gene products of the specific genes can increase or decrease due to the presence of a neurodegenerative disease. Then, a test substance with similar efficacy to edaravone can increase or decrease the expression levels of the gene products of the specific genes. Therefore, when no change in expression level of a gene product in a target that has been exposed to a test substance is confirmed compared to an expression level of a gene product in a target that has been exposed to edaravone for one or more specific genes, the test substance can be selected as a substance capable of treating or preventing a neurodegenerative disease. Further, it can also be determined that the test substance has a high possibility of being a substance having efficacy equivalent to edaravone in treating or preventing a neurodegenerative disease.

On the other hand, when a change in expression level of a gene product in the test substance-exposed group is confirmed compared to an expression level of a gene product in the edaravone-exposed group for one or more specific genes, it can be determined that the test substance has a low possibility of being a substance capable of treating or preventing a neurodegenerative disease. Further, it can be determined that the test substance does not have efficacy equivalent to edaravone, or has a low possibility of having efficacy, in treating or preventing a neurodegenerative disease.

Further, for example, for one or more genes belonging to the gene group (A) among the specific genes, when an expression level of a gene product in the test substance-exposed group is decreased compared to an expression level of a gene product in the unexposed group and is increased compared to an expression level of a gene product in the edaravone-exposed group, it can be evaluated that there is a possibility that the efficacy of the test substance is less than the efficacy of edaravone. For the gene belonging to the gene group (B) among the specific genes, when an expression level of a gene product in the test substance-exposed group is increased compared to an expression level of a gene product in the unexposed group and is decreased compared to an expression level of a gene product in the edaravone-exposed group, it can be evaluated that there is a possibility that the efficacy of the test substance is less than the efficacy of edaravone. Based on such evaluations, measures can be taken to enhance the efficacy, such as changing the test substance used or adjusting the exposure amount or exposure frequency of the test substance.

In any case, the determination of the change in expression level is preferably performed based on the presence or absence of a statistically significant difference from a point of view of improving the determination accuracy.

In one embodiment, in order to determine in the process B3 that a test substance is an intended substance, for example, the determination can also be performed by comparing a result of an expression level in the test substance-exposed group and a result of an expression level in a normal target.

Specifically, in one embodiment, the process B3 preferably includes at least one of the following processes B36, B37 and B38.

In one embodiment, the present method can include, for example, performing at least one of the processes B37 and B38 after the process A3.

In one embodiment, the present method can include, for example, performing the process B36 before or after the process A3, or simultaneously with the process A3, and then performing the processes B37 and B38 in this order.

In one embodiment, the present method may adopt a combination of two or all of the processes B31, B32 and B33 described above, or a combination of the processes B34 and B35 described above.

The process B36 is a process of measuring an expression level of a gene product of a specific gene in a control group, using a normal target that has not been exposed to a test substance and edaravone as the control group.

The process B37 is a process of comparing an expression level of a gene product in a normal target (control group) with an expression level of a gene product in a target that has been in contact with a test substance and has or is suspected of having a neurodegenerative disease (test substance-exposed group).

The process B38 is a process of comparing a normal target (control group) and an exposed group, and when no change in expression level of a gene product is confirmed for one or more specific genes, determining that a test substance is a substance capable of treating or preventing a neurodegenerative disease and selecting the substance.

The determination of the change in expression level may be performed by setting an arbitrary threshold or may be performed based on a statistically significant difference. The determination of the change in expression level is preferably performed based on the presence or absence of a statistically significant difference from a point of view of improving the determination accuracy.

An expression level of a gene product of one or more genes among the specific genes described above can increase or decrease due to the presence of a neurodegenerative disease. Therefore, when there is a possibility that a test substance is a substance capable of treating or preventing a neurodegenerative disease, an expression level of a gene product of one or more genes among these specific genes in a test substance-exposed group can decrease or increase.

Therefore, when no change in expression level of a gene product in a test substance-exposed group is confirmed compared to an expression level in a normal target for one or more specific genes, it can be determined that the test substance is a substance capable of treating or preventing a neurodegenerative disease.

On the other hand, when a change in a gene product in a test substance-exposed group is confirmed compared to an expression level in a normal target for one or more specific genes, it can be determined that the test substance is not a substance capable of treating or preventing a neurodegenerative disease or such a possibility is low.

Gene products of the specific genes described above can also be used as biomarkers. That is, the present embodiment relates to biomarkers.

As shown in the examples to be described below, the specific genes described above were newly identified as those showing a change in expression level based on results from exposure to edaravone and disease models. Therefore, by using a gene product of a specific gene as a biomarker, it is possible to easily diagnose the presence or absence of a neurodegenerative disease, the progression or severity of the disease, or the possibility of the disease. Further, by using a gene product as a biomarker, it is possible to easily evaluate the possibility of contributing to the responsiveness to or efficacy of edaravone against a disease, or the efficacy of a test substance.

Specifically, in one embodiment, a biomarker includes a gene product of one or more of specific genes selected from KAZALD1, SBK1, SCN2A, UBE2L6, ALPL, NTM, PTTG1, ITGB4, HAUS4, DCTD, MT2A, ASF1B, FCSK, MAST1, and FAIM2. By including multiple types of gene products derived from two or more genes as a biomarker, the reliability of evaluation or diagnosis can be improved.

In one embodiment, the biomarker described above may consist solely of a gene product derived from a specific gene described above.

A gene product in the biomarker described above may consist solely of a transcription product, may consist solely of a translation product, or may consist of a combination of a transcription product and a translation product.

The biomarker described above can be used to determine the presence or possibility of a neurodegenerative disease in a target, or to predict prognosis.

In another embodiment, the biomarker described above is used for prediction of responsiveness to edaravone.

In yet another embodiment, the biomarker described above is used for diagnosing responsiveness to a test substance.

For the biomarker, one or more types of the samples described above, more specifically, tissues, cells, and body fluids collected from living organisms such as animals regardless of whether or not edaravone has been administered, and animals regardless of whether or not they have a disease, can be used as a specimen, and at least one of a transcription product and a translation product in the specimen can be measured using the measurement method described above. Preferably, this can be performed in vitro.

A detection kit for detecting the biomarker according to an embodiment of the present invention includes a detection reagent capable of specifically detecting a gene product of a specific gene. This kit can detect, preferably in vitro, one or more of the gene products as a biomarker.

Examples of the detection reagent include a nucleic acid probe or primer for detecting a transcription product of a specific gene. Other examples of the detection reagent include an antibody or antibody fragment or the like for detecting a translation product of a specific gene. The detection reagent may be labeled, for example, with a fluorescent substance or a radioactive nuclide, when necessary.

Further, a method for treating a neurodegenerative disease using the composition described above according to an embodiment of the present invention may include, for example, orally or parenterally administering the composition described above to a human or a non-human animal. When an administration target is a human, the human is preferably a patient with a neurodegenerative disease.

In one embodiment, the treatment method described above can adopt administration conditions using, for example, edaravone injection or oral administration formulations as an edaravone-containing composition. For administration conditions of edaravone injection, for example, the administration method described in WO2020/091036, or the administration method using edaravone preparations used in clinical settings can be adopted. The entire contents of this publication are incorporated herein by reference.

Further, an embodiment of the present invention provides the use of a substance that causes a change in expression level of a gene product of the specific genes described above in the manufacture of a composition for the prevention or treatment of a neurodegenerative disease.

Further, an embodiment of the present invention provides a substance for use in causing a change in expression level of a gene product of the specific genes described above. In one embodiment, a substance is also provided for use in the treatment of a neurodegenerative disease by causing a change in expression level of a gene product of the specific genes described above.

The substance described above is not particularly limited, and examples thereof include edaravone, edaravone-containing compositions, organic low molecular weight compounds, nucleic acids, proteins, peptides, antibodies, natural components, and test substances found using the screening method described above.

An embodiment of the present invention provides a method for suppressing the occurrence or progression of cellular damage by causing a change in expression level of a gene product of the specific genes described above in cells.

For causing a change in expression level, for example, a method for bringing cells into contact with a predetermined substance can be used. The substance used for the contact is not particularly limited, and examples thereof include edaravone, edaravone-containing compositions, organic low molecular weight compounds, nucleic acids, proteins, peptides, antibodies, natural components, and test substances found using the screening method described above.

The above-described embodiments described in the present specification may each independently adopt one of the specific genes described above, or all of the specific genes, or any combination of two or more of the specific genes. The following combinations of the specific genes are examples, and it is not limited to these combinations.

For example, in one embodiment, the specific genes described above may be one or more selected from KAZALD1, SBK1, UBE2L6, ALPL, NTM, PTTG1, ITGB4, HAUS4, DCTD, MT2A, ASF1B, FCSK, MAST1 and FAIM2.

Further, for example, in one embodiment, the specific genes described above may be one or more selected from KAZALD1, SBK1, UBE2L6, NTM, HAUS4, DCTD, ASF1B, FCSK, and FAIM2.

Further, for example, in one embodiment, the specific genes described above may be one or more selected from KAZALD1, SBK1, UBE2L6, NTM, DCTD, ASF1B and FCSK.

Further, for example, in one embodiment, the specific genes described above may be one or more selected from KAZALD1, SBK1, DCTD and FCSK.

Further, for example, in one embodiment, the specific genes described above may be one or more selected from SBK1, DCTD and FCSK.

Further, for example, in one embodiment, the specific genes described above may be one or two selected from DCTD and FCSK.

### EXAMPLES

In the following, the present invention is further described in detail based on examples. However, the scope of the present invention is not limited to such examples.

### Example 1: Preparation of Disease Model Cells

### (1): Cell Culture and Differentiation

As a target, a 1464R adult rat neural stem cell line (hereinafter also referred to as the “1464R cell line” or “undifferentiated cell line”) was used. The 1464R cell line was established using a method described in Neuropathology 2014, 34, 83-98. The entire contents of this publication are incorporated herein by reference.

The cell line was cultured in a non-adherent state in maintenance medium using a 10 cm dish treated with poly-2-hydroxyethyl methacrylate. Culture conditions were 37° C. in a 5% CO₂ environment, with subculturing performed twice a week.

The maintenance medium was prepared to contain the following components at their final concentrations in a Neurobasal Plus medium (manufactured by Thermo Fisher Scientific).

Typical neurosphere-forming 1464R cells were isolated. The isolated 1464R cells were dispersed and maintained in culture.

Additives in Maintenance Medium
2 mM L-Glutamine (manufactured by Thermo Fisher Scientific)
2% v/v B-27 Supplement (manufactured by Thermo Fisher Scientific)
10 ng/mL Fibroblast Growth Factor (FGF)-2 (manufactured by Sigma)
10 ng/mL Epidermal Growth Factor (EGF) (manufactured by Sigma)
50 units/mL Penicillin and 50 μg/mL Streptomycin (manufactured by Thermo Fisher Scientific)

Next, using a differentiation medium shown below, the 1464R cell line was cultured in an adherent state on a poly-L-lysine treated dish. Culture conditions for cell differentiation were 37° C., 5% CO₂, and 4 days. Through this process, the 1464R cell line was differentiated into neuronal cells (neurons). Further, the 1464R cell line partly differentiated into glial cells. In the following description, the differentiated 1464R cell line is also collectively referred to as the “differentiated cell line.”

The differentiation medium was prepared to contain the following components at their final concentrations in an F-12 medium (manufactured by Thermo Fisher Scientific).

### Additives in Differentiation Medium

5 v/v % Fetal Bovine Serum (manufactured by Moregate)
0.5 v/v % N-2 supplement (manufactured by Thermo Fisher Scientific)
1 v/v % B-27 supplement (manufactured by Thermo Fisher Scientific)
1 μmol/L ATRA (manufactured by Sigma)
50 units/mL Penicillin and 50 μg/mL Streptomycin (manufactured by Thermo Fisher Scientific)

### (2): Forced Expression of TDP-43 Protein

Subsequently, a neurodegenerative disease in vitro model was prepared by forcibly and excessively expressing TDP-43 protein in the differentiated cell line according to the following method.

After obtaining the differentiated cell line using the method described in (1), the differentiation medium was removed and replaced with infection medium. The composition of the infection medium was prepared to contain the following components at their final concentrations in F-12 medium (manufactured by Thermo Fisher Scientific) without an antioxidant and serum: The culture conditions were maintained at 37° C. in a 5% CO₂ environment.

### Additives in Infection Medium

0.5 v/v % N-2 supplement (manufactured by Thermo Fisher Scientific)
1 μmol/L ATRA (manufactured by Sigma)
50 units/mL Penicillin and 50 μg/mL Streptomycin (manufactured by Thermo Fisher Scientific)

Separately, the following recombinant adenovirus vectors (a) and (b) were prepared according to a method described in Neuropathology 2014, 34, 83-98.
(a) Wild-type: Recombinant adenovirus vector containing cDNA encoding full-length human wild-type TDP-43 labeled with DsRed (AxDsRhWTTDP43 strain; RIKEN DNA Bank Japan; #RDB15499)
(b) Mutant: Recombinant adenovirus vector containing cDNA encoding the C-terminal fragment of TDP-43 (amino acid sequence numbers 208-414) labeled with DsRed (AxDsRhWTTDP43 strain; RIKEN DNA Bank Japan; #RDB15500)

Using both vectors (a) and (b), the differentiated cell line in the infection medium was infected with adenovirus at a multiplicity of infection of 50, and cultured for 24 hours. As a result, neurons derived from the 1464R cell line, in which both wild-type and mutant TDP-43 were forcibly expressed intracellularly (hereinafter, also referred to as disease model cells), were obtained.

These disease model cells serve as an in vitro model of a neurodegenerative disease, in which TDP-43 expression localization and the presence of mutant TDP-43 protein may be involved. An example of such a neurodegenerative disease is ALS.

### Example 2: Evaluation of Cell Protection by Microscopic Observation

Whether or not cellular damage is suppressed by edaravone (hereinafter also referred to as Eda) was evaluated using the disease model cells prepared using the method of Example 1.

Using a 96-well plate treated with poly-L-lysine, cells were seeded at a concentration of 8×10⁴ cells/well, cultured and differentiated under the conditions of (1) in Example 1. After that, using the method of (2) in Example 1, TDP-43 was forcibly expressed in differentiated cells to obtain disease model cells.

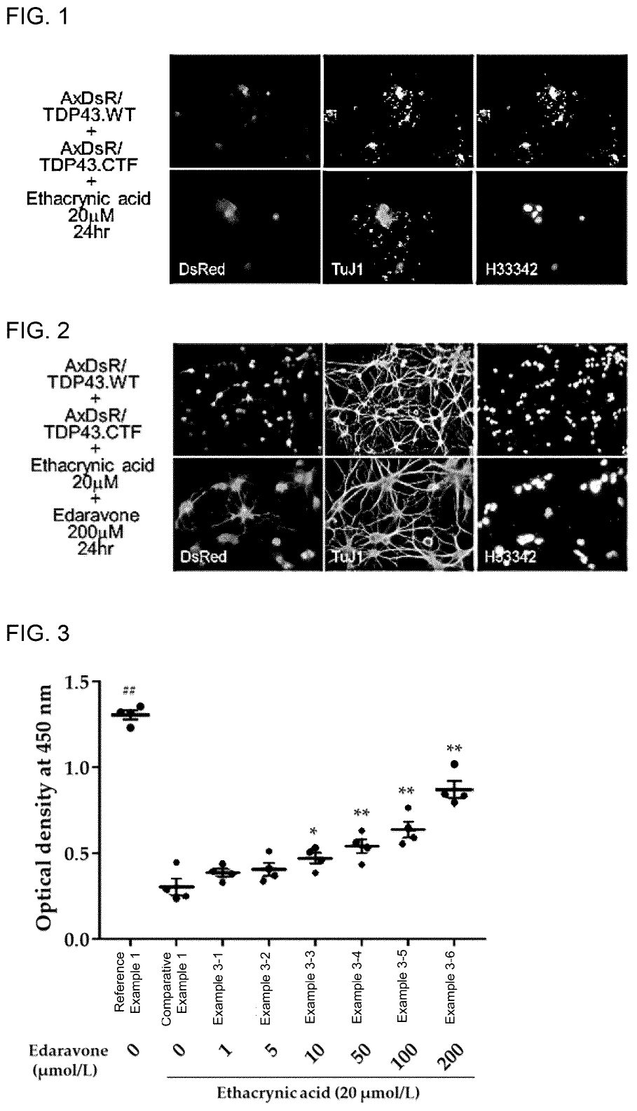

For the disease model cells, as an Eda-free group, a group exposed to EA (final concentration 20 μmol/L) for 24 hours was prepared (See FIG. 1). Separately, as an Eda-containing group, a group exposed to Eda (final concentration 200 μmol/L) for 24 hours and then exposed to EA (final concentration 20 μmol/L) for 24 hours was prepared (See FIG. 2). Then, cell morphology in each group was observed using a fluorescence microscope.

Tubulin β III (represented by TuJ1 in the figure), an indicator of neuronal differentiation, was fluorescently stained by fixing cells after 24 hours of EA exposure with 4% paraformaldehyde, treating them with TuJ1 antibody overnight at 4° C., and then treating them with fluorescent antibody for 15 minutes at room temperature. Cell nuclei were fluorescently stained using Hoechst 33342 using a conventional method. The results are shown in FIGS. 1 and 2.

As shown in FIG. 1, in the Eda-free group, it was confirmed that the neuron-specific protrusion shape observed by the localization of tubulin BIII had disappeared, indicating cellular damage had occurred.

On the other hand, as shown in FIG. 2, in the Eda-containing group, it was confirmed that the neuron-specific protrusion shape observed by the localization of tubulin BIII was sufficiently maintained, indicating a cell-protective effect. The localization of TDP-43 confirmed by DsRed, and the localization of cell nuclei were also confirmed to be present within the cells.

### Examples 3-1 to 3-6, Comparative Example 1, and Reference Example 1: Evaluation of Cell Protection Using Chromogenic Substrate as Indicator

Disease model cells prepared using the method of Example 1 were used to prepare the following groups: Eda-containing groups (Examples 3-1 to 3-6): the cells were exposed to Eda (final concentration 1-200 μmol/L) for 24 hours, followed by exposure to EA (final concentration 20 μmol/L) for 24 hours; Eda-free group (Comparative Example 1): the cells were exposed to EA (final concentration 20 μmol/L) for 24 hours without prior exposure to Eda; and unexposed group (Reference Example 1): the cells were not exposed to either Eda or EA.

After that, a cell toxicity measurement kit (Cell Counting Kit-8, manufactured by DOJINDO) was used to expose the cells in each group according to a product protocol, and an amount of chromogenic substrate produced was measured by absorbance at 450 nm.

With this kit, the more viable cells present, the more chromogenic substrate is produced, resulting in a higher absorbance value at 450 nm. Therefore, a higher absorbance at 450 nm indicates a greater number of viable cells. The results are shown in FIG. 3.

The data shown in FIG. 3 are all expressed as mean±standard error of the mean (SEM, n=4 for each). In FIG. 3, the symbol “##” indicates a statistically significant difference of p<0.01 compared to Comparative Example 1 (determined by Student t-test). In FIG. 3, the symbol “*” indicates a statistically significant difference of p<0.05 compared to Comparative Example 1, and the symbol “**” indicates a statistically significant difference of p<0.01 compared to Comparative Example 1 (both determined by Williams' multiple comparison test).

As shown in FIG. 3, in the Eda-containing groups (Examples 3-1 to 3-6), compared to the Eda-unexposed group (Comparative Example 1), the absorbance at 450 nm increased in an Eda concentration-dependent manner, confirming a higher number of viable cells. This indicates that cellular damage was suppressed in the presence of Eda. Further, similar results to those shown in FIG. 3 were obtained even when the disease model cells prepared using the method of Example 1 were simultaneously exposed to both Eda and EA for 24 hours.

### Example 4: Exploration and Evaluation of Biomarkers

RNA-seq analysis was performed using the disease model cells prepared using the method of Example 1 to identify biomarkers that could serve as indicators for disease progression, evaluation of treatment efficacy, and/or selection of candidate compounds.

In the present evaluation, a 6-well plate treated with poly-L-lysine was used, cells were seeded at a density of 1×10⁶ cells per well, cultured under the conditions of (1) of Example 1, and differentiated cells were used.

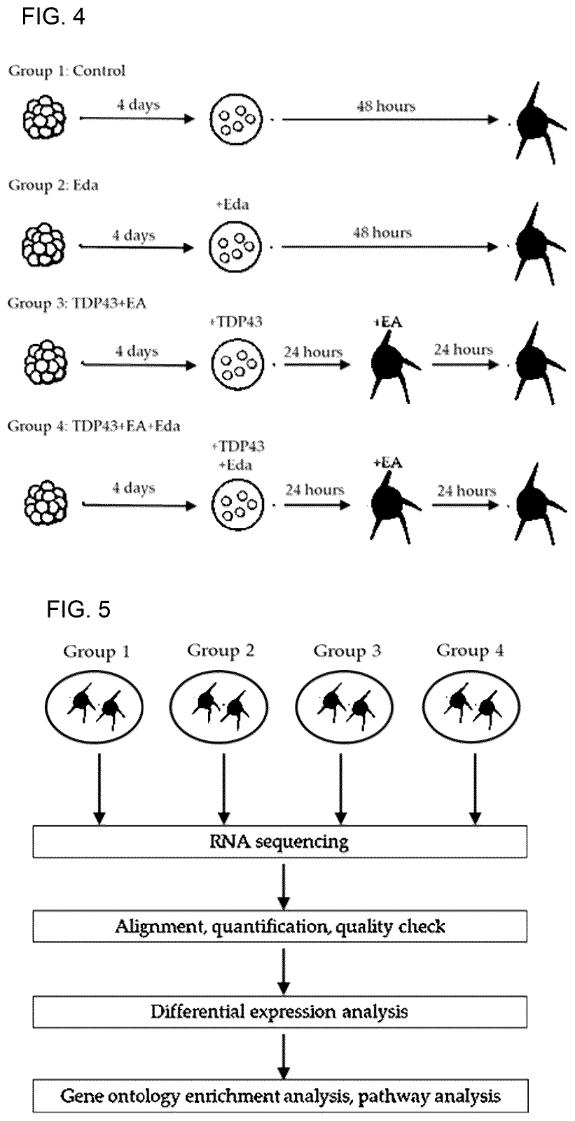

In the following, experimental groups (1) to (4) of the present example are shown along with FIG. 4.

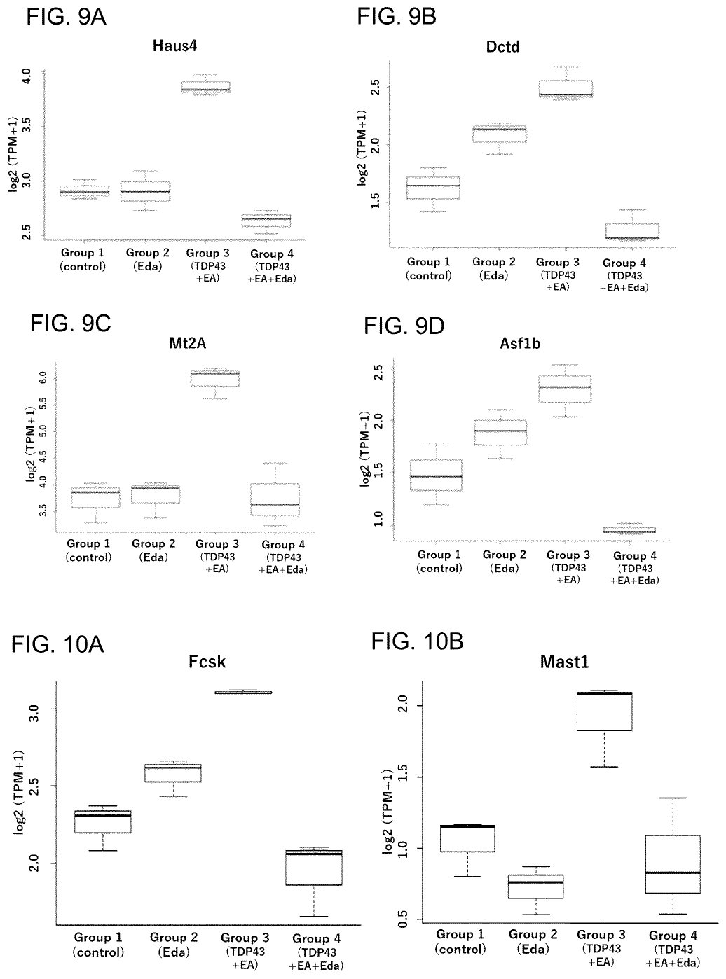

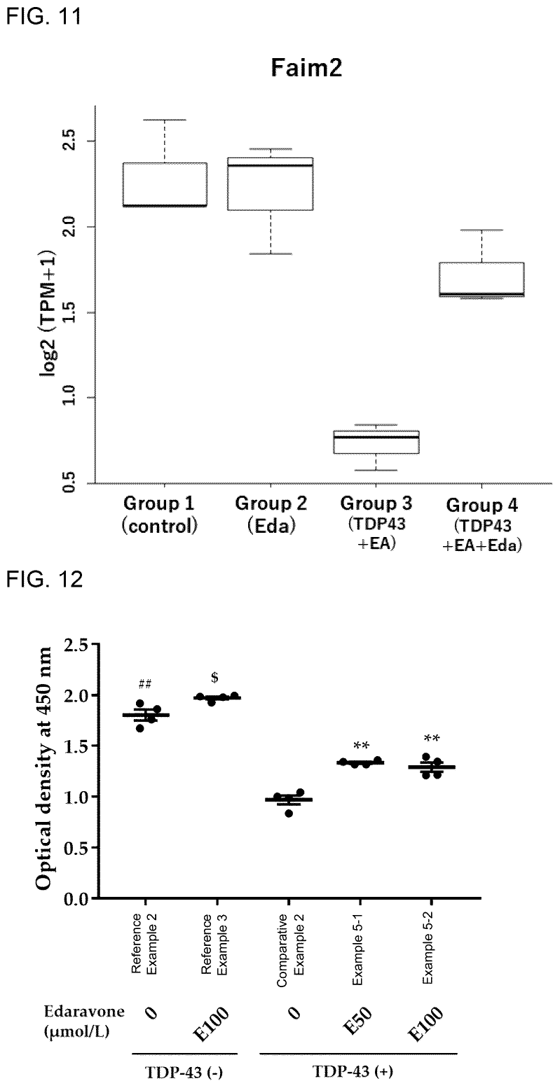

(1) Group 1 (control group; represented as “Group 1” in FIG. 4 and FIGS. 7-11): After preparing the differentiated cell line using the method of (1) of Example 1, the cells were further cultured in Eda and EA-free differentiation medium for 48 hours. This experimental group does not forcibly express TDP-43 and simulates healthy animals, including humans.

(2) Group 2 (Eda-only exposed group; represented as ‘Group 2’ in FIG. 4 and FIGS. 7-11): After preparing the differentiated cell line using the method of (1) of Example 1, the cells were cultured in differentiation medium containing Eda (final concentration 50 μmol/L) but free of EA for 48 hours. This experimental group does not forcibly express TDP-43 and simulates conditions in which healthy animals, including humans, are exposed to Eda.

(3) Group 3 (TDP-43+EA exposed group; represented as ‘Group 3’ in FIG. 4 and FIGS. 7-11): After preparing the disease model cells expressing TDP-43, the cells were cultured in differentiation medium free of Eda but containing EA (final concentration 20 μmol/L) for 24 hours. This experimental group forcibly expresses TDP-43 and simulates conditions where the target has a neurodegenerative disease and remains untreated.

(4) Group 4 (TDP-43+EA+Eda exposed group; represented as ‘Group 4’ in FIG. 4 and FIGS. 7-11): After preparing the disease model cells expressing TDP-43, the cells were cultured in differentiation medium containing Eda (final concentration 50 μmol/L) but free of EA for 24 hours. After that, EA (final concentration 20 μmol/L) was further added, and the cells were further cultured for an additional 24 hours. This experimental group simulates conditions where the target with a neurodegenerative disease is administered or exposed to Eda.

After the culture was completed, cell pellets containing 1×10⁶ or more cells were obtained from the experimental groups (1)-(4). Total RNA was extracted from these cell pellets using NucleoSpin RNA (manufactured by Macherey-Nagel) according to a product protocol.

After that, using TapeStation and BioanalizerRNA6000 Nano Chip (manufactured by Agilent), it was confirmed that all of the extracted RNAs were of high quality, with an RNA concentration of 2 ng/μL or more and a total RNA amount of 50 ng or more.

Using the extracted total RNA (1.0 ng) as a template, double-stranded cDNA was synthesized using the SMART method using the SMART-Seq v4 Ultra (registered trademark) Low Input RNA Kit for Sequencing (manufactured by Clontech) according to a product protocol.

After that, the synthesized double-stranded cDNA was amplified by performing 13 cycles of PCR according to a product protocol. Then, a PCR product was purified using a magnetic bead method using AMPureXP (manufactured by Beckman Coulter).

Using the Nextera XT DNA Library Prep Kit and Nextera XT Index Kit v2 (manufactured by Illumina), the obtained PCR product (double-stranded cDNA) was tagmented and both ends of a fragmented cDNA were ligated with adapter sequences according to a product protocol. After that, the ligated cDNA (0.2 ng) was amplified by 12 cycles of PCR using a primer provided in the kit according to a product protocol. Then, the PCR products of all samples were pooled into one library to avoid any adverse effects that may occur during sequencing.

The PCR products obtained from the experimental groups were processed in several processes as shown in FIG. 5.

### RNA Sequencing (See FIG. 5)

Next, using the NovaSeq6000 system (NovaSeq6000 S4 reagent kit, NovaSeq Xp4 lane kit, and NovaSeq control software (version 1.6.0); all manufactured by Illumina), sequences of the cDNA library were determined according to a product protocol.

Base calls and quality scores were calculated using real-time analysis (version 3.4.4; manufactured by Illumina) according to cycle-based call files. The base call files were converted to FASTQ files using bcl2fastq2 software (version 2.20, manufactured by Illumina).

### Alignment—Expression Analysis (see FIG. 5)

Next, using the DRAGEN Bio-IT platform software (version 3.6.3, manufactured by Illumina) and referring to the rat genome assembly (version 6.0, top-level) and transcript annotation (release 101) obtained from the Ensembl database, types and abundance of genes were identified from the 150-base sequence information contained in the FASTQ files mentioned above.

An RNA expression level (TPM) of a transcription product i measured from the cDNA library of the experimental groups was calculated based on alignment to the rat genome and transcription product annotation from the Ensembl database, according to the following equation (A).

In the following equation (A), “m_i” represents the number of reads for the transcription product i, and “I_i” represents the base length of the transcription product i.

TPM_i=10⁶×(m_i/1_i)/(Σ_i[(m_i/1_i)]) (A)

Differential expression analysis was performed using DESeq2 (a package for two-group comparison) in the statistical analysis software R (version 3.6.0) to calculate a log 2 ratio of RNA expression levels and a significance test. The log 2 ratio is a value expressed as “log 2 ([expression level of a first measurement target]/[expression level of a second measurement target]).”

The significance test was performed based on a p-value calculated by Wald test with multiple testing correction based on the Benjamini-Hochberg method (BH method).

In the present analysis, a gene with an absolute log 2 ratio of 1 or more (expression change ratio of 2.0 times or more or 0.50 times or less) and a p-value of 0.01 or less from the Wald test between any two groups was identified as a “gene with a change in expression level” (differentially expressed gene; DEG). DEGs include both upregulated genes and downregulated genes.

### Gene Ontology Analysis (See FIG. 5)

Gene ontology analysis was performed by associating DEGs with ontology terms obtained from the BioMart database. These associations were performed by considering an ontology term with a p-value of 0.05 or less in the hypergeometric distribution test with multiple testing correction based on the BH method as significant. Classification of gene ontology related to DEGs was performed focusing on biological function, structural component, and molecular function, and significant ontology terms were extracted.

### Expression Regulatory Pathway Analysis (See FIG. 5)

Expression regulatory pathway analysis of DEGs was performed using software (Ingenuity Pathway Analysis, manufactured by QIAGEN). Association between expression pathways defined in the software and DEGs was performed by considering those with a p-value of 0.05 or less calculated by Fisher's exact test with multiple testing correction based on the BH method as significant.

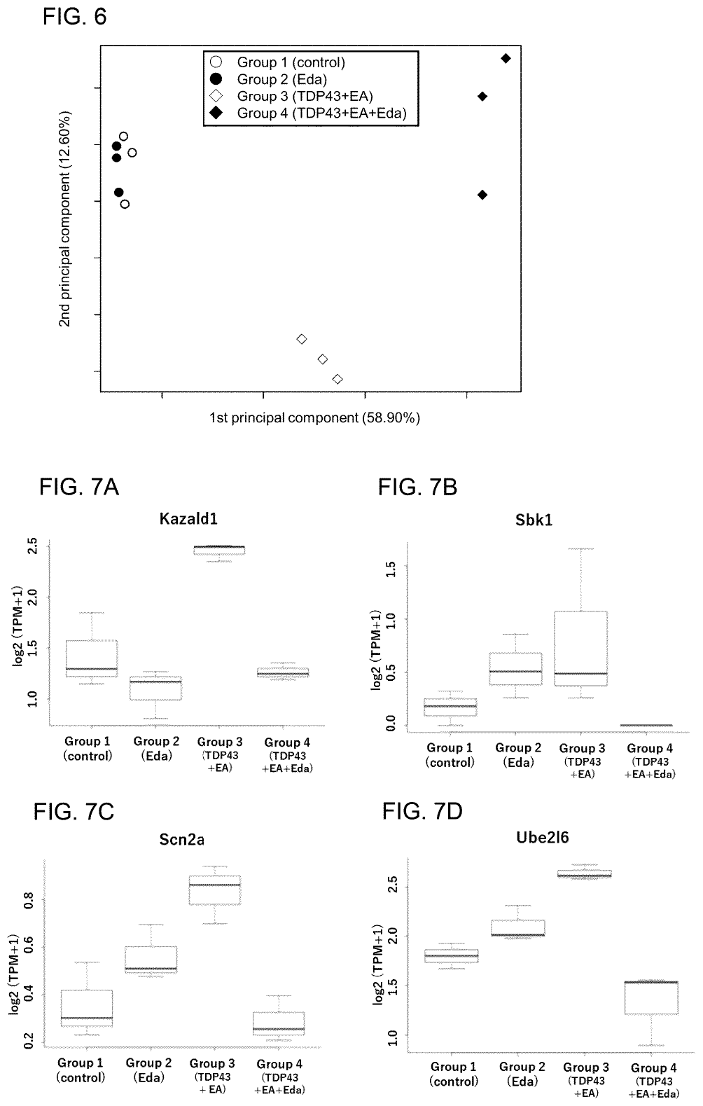

### Analysis Results in Present Example are Shown in FIG. 6.

FIG. 6 shows results of principal component analysis of expression changes in RNA-seq data obtained from the experimental groups (N=3 for each). The X-axis represents a first principal component, and the Y-axis represents a second principal component, with their respective contribution rates expressed as percentages.

The principal component analysis was performed by excluding genes with a TPM variance value of 1 or less across all samples.

As shown in FIG. 6, the RNA-seq data of samples from the same experimental group were all close to each other, indicating good quality of the RNA-seq data.

Comparing the experimental groups, it was found that Groups 3 and 4 had clearly different gene expression profiles compared to Group 1.

Further, it was found that Groups 3 and 4 had distinctly different gene expression profiles, despite both expressing TDP-43.

On the other hand, it was found that Groups 1 and 2 had similar gene expression profiles, regardless of the presence or absence of Eda exposure.

Table 1 below shows the results of identifying differentially expressed genes (DEGs) between the experimental groups based on the gene expression levels of the groups. In Table 1, “xxx/yyy” (where xxx and yyy independently represent any integers) indicates that the number of upregulated genes is xxx and the number of downregulated genes is yyy.

TABLE 1

| Group | Group 1 | Group 2 | Group 3 | Group 4 |
|---|---|---|---|---|
| Control (Group 1) | — | 0/0 | 853/467 | 1,654/1,095 |
| Eda (Group 2) | — | — | 876/478 | 1,661/1,130 |
| TDP43 + EA | — | — | — | 169/260 |
| (Group 3) | | | | |
| TDP43 + EA + Eda | — | — | — | — |
| (Group 4) | | | | |

From the differentially expressed genes in the experimental groups, genes meeting either criterion a or criterion b below were identified, and their expression change rates are shown in FIGS. 7 to 11.

Criterion a: Significantly upregulated in Group 3 compared to Group 1, and significantly downregulated in Group 4 compared to Group 3.

Criterion b: Significantly downregulated in Group 3 compared to Group 1, and significantly upregulated in Group 4 compared to Group 3.

A gene that meets any of the above criteria means that its expression level changes with the onset of a disease (corresponding to Group 3) compared to that in a healthy state (corresponding to Group 1), and that its expression level returns to a level equivalent to or close to normal by treatment of the disease (corresponding to Group 4).

Therefore, the expression levels of these genes can serve as useful biomarkers, for example, by using changes in the expression levels as indicators, for: evaluating or predicting the possibility of the presence or absence of a disease and a degree of progression of the disease; evaluating or predicting the possibility of treatment responsiveness; selecting appropriate specimens for use as disease models; or selecting candidate compounds during drug discovery.

The genes shown in FIGS. 7 to 11 are all rat genes that meet the above criteria and have human homologues.

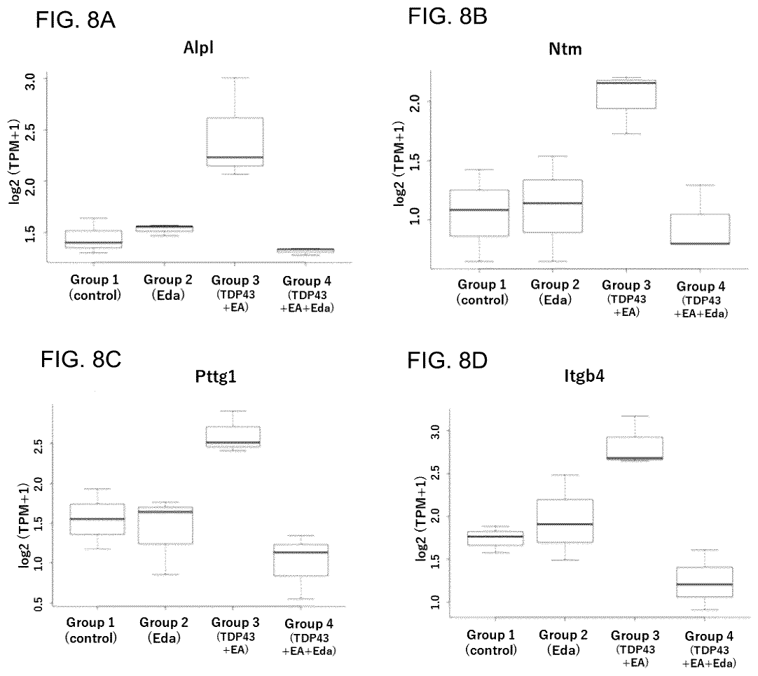

As an example, Alpl (rat ortholog of human ALPL) shown in FIG. 8 (a) is a gene that encodes alkaline phosphatase. Alkaline phosphatase is an enzyme present in extracellular fluids such as blood, and thus, is advantageous, for example, for evaluating disease progression or treatment effectiveness or assisting in such predictions in a minimally invasive manner by collecting extracellular fluids such as blood.

Tables 2 and 3 below show the results of gene ontology analysis, as well as the differentially expressed genes, their expression change amounts (TPM), expression change log 2 ratios, and p-values.

Further, Table 4 below shows the expression change log 2 ratios and p-values for other differentially expressed genes, which are rat genes that meet the criteria a and b described above. All the genes listed in Table 4 are included in the specific genes according to an embodiment of the present invention.

In Table 4, “Upregulated genes” are examples of genes whose expression levels are decreased by the presence of disease and increased by the presence of disease and edaravone. These genes are included in the gene group (B) described above.

In Table 4, “Downregulated genes” are examples of genes whose expression levels are increased by the presence of disease and decreased by the presence of disease and edaravone. These genes are included in the gene group (A) described above.

For convenience of description, the results for the Mt2A and Alpl genes listed in Tables 2 and 3 are re-presented in Table 4. Quantitative PCR for Mt2A and Alpl is performed and expression profiles similar to those based on RNA-seq were obtained.

TABLE 2
<DEG> Group3: Up/Group 4: Down

| Gene | | Oncology | | TPM | | |
|---|---|---|---|---|---|---|
| Name | Description | Biological.Process.Name | Molecular.Function.Name | Group 1 | Group 2 | Group 3 |
| [symbol] | [symbol] | [symbol] | [symbol] | 1.4317 | 1.083 [symbol] | [symbol] .4495 |
| [symbol] | [symbol] | [symbol] | [symbol] | 0.1885 | 0. [symbol] 392 | 0.8017 |
| [symbol] | [symbol] | [symbol] | [symbol] | 0.3831 | 0.8 [symbol] 7 | 0.8328 |
| [symbol] | [symbol] | [symbol] | [symbol] | 1.8012 | 2.100 [symbol] | 2.6283 |
| [symbol] | [symbol] | [symbol] | [symbol] | 1.4457 | 1.5 [symbol] 09 | 2.433 [symbol] |
| [symbol] | [symbol] | — | — | 1.0515 | 1.1081 | 2.02 [symbol] 8 |
| [symbol] | [symbol] | [symbol] | [symbol] | 1.55 [symbol] | 1.4190 | 2.80 [symbol] 5 |

| | | p value | log 2 ratio | p value | log 2 ratio |
|---|---|---|---|---|---|
| Gene | TPM | Group 3 vs | Group 3/ | Group 4 vs | Group 4/ |
| Name | Group 4 | Group 1 | Group 1 | Group 3 | Group 3 |
| [symbol] | 1.2640 | 0.0008126214 | 1. [symbol] 8902 [symbol] 44 | 0.0010879 [symbol] 49 | −1.1087 [symbol] 026 [symbol] 9 |
| [symbol] | 0.0000 | 0.007 [symbol] 354 [symbol] | 1.4 [symbol] 9 [symbol] 92 [symbol] 89 | 0.00000 [symbol] | −1.826227577 [symbol] |
| [symbol] | 0.2 [symbol] 7 [symbol] | 0.00 [symbol] 67791 [symbol] 8 | 1.141 [symbol] | 0.000 [symbol] | −1.206 [symbol] 23 [symbol] 72 |
| [symbol] | 1.2231 | 0.001674614 [symbol] | 1. [symbol] 3797 [symbol] 341 | 0.0029075 [symbol] 7 | −1.0 [symbol] 207 [symbol] 56 [symbol] |
| [symbol] | 1.3164 | 0.00013922 [symbol] 4 | 1.3 [symbol] 429948 [symbol] 0 | 0.00105 [symbol] 8103 | −1.0975312910 |
| [symbol] | 0. [symbol] 20 | 0.0003320477 | 1.35 [symbol] 783106 | 0.0011423348 | −1.11 [symbol] 7 [symbol] 49 [symbol] |
| [symbol] | 1.0105 | 0. [symbol] | 1.19 [symbol] 018 [symbol] 219 | 0.001 [symbol] 0 [symbol] 14 | −1.2 [symbol] 9 [symbol] 052930 |

[symbol]

indicates data missing or illegible when filed

TABLE 3

| Gene | | Oncology | | TPM | | |
|---|---|---|---|---|---|---|
| Name | Description | Biological.Process.Name | Molecular.Function.Name | Group 1 | Group 2 | Group 3 |

<DEG> Group3: Up/Group 4: Down

| | | | | | | |
|---|---|---|---|---|---|---|
| [symbol] | [symbol] | [symbol] | [symbol] | 1. [symbol] 4 [symbol] | 1. [symbol] 811 | 2. [symbol] |
| [symbol] | [symbol] | [symbol] | [symbol] | [symbol] .9144 | 2.9 [symbol] 66 | 3.867 [symbol] |
| [symbol] | [symbol] | [symbol] | [symbol] | 1.6212 | [symbol] .080 [symbol] | 2.504 [symbol] |
| [symbol] | [symbol] | [symbol] | [symbol] | 3.724 [symbol] | 3.78 [symbol] 8 | 5. [symbol] |
| [symbol] | [symbol] | [symbol] | [symbol] | 1.4827 | 1.87 [symbol] | 2.2951 |
| [symbol] | [symbol] | [symbol] | [symbol] | 2.2627 | 2.5895 | 3.1070 |
| MAST1 | [symbol] | [symbol] | [symbol] | 1. [symbol] | 0.72 [symbol] 3 | 1.92 [symbol] 8 |

<DEG> Group3: Down/Group 4: Up

| | | | | | | |
|---|---|---|---|---|---|---|
| [symbol] | [symbol] | — | — | 2.2 [symbol] 13 | 2.2 [symbol] 2 | 0.7312 |

| | | p value | log 2 ratio | p value | log 2 ratio |
|---|---|---|---|---|---|
| Gene | TPM | Group 3 vs | Group 3/ | Group 4 vs | Group 4/ |
| Name | Group 4 | Group 1 | Group 1 | Group 3 | Group 3 |
| <DEG> Group3: Up/Group 4: Down | | | | | |
| [symbol] | 1.24 [symbol] 7 | 0.0000000001 | 1.3 [symbol] 4 [symbol] 68 [symbol] | 0.000000000 [symbol] | −1. [symbol] 036 [symbol] 77 [symbol] |
| [symbol] | 2. [symbol] 31 [symbol] | 0.0000001849 | 1.11032 [symbol] 91 [symbol] | 0.0000069443 | −1. [symbol] 3928069 [symbol] 7 |
| [symbol] | 1.2538 | 0.0003 [symbol] 102 [symbol] 0 | 1.115 [symbol] 032 [symbol] 3 | 0.0001649481 | −1.15724437 [symbol] |
| [symbol] | 3.7584 | 0.0000000 [symbol] | 2.1 [symbol] | 0.0000 [symbol] 11021 | −1.424978179 [symbol] |
| [symbol] | 0.9583 | 0.0079022897 | 1. [symbol] 47 [symbol] | 0.00024 [symbol] 8304 | −1.322 [symbol] 4 [symbol] 36 [symbol] |
| [symbol] | 1.9 [symbol] 93 | 0.0000000027 | 1.08181 [symbol] 7 | 0.0000047070 | −1.04723441 [symbol] 1 |
| MAST1 | 0.90 [symbol] 4 | 0.000 [symbol] 118128 | 1. [symbol] | 0.00 [symbol] 1 [symbol] 078 | −1.05218138 [symbol] |
| <DEG> Group3: Down/Group 4: Up | | | | | |
| [symbol] | 1.72 [symbol] 0 | 0.000004 [symbol] 024 | −1. [symbol] 3 [symbol] 4 [symbol] 10 | 0.00120932 [symbol] 0 | 1.2684526238 |

[symbol]

indicates data missing or illegible when filed

| Gene | | Group 3 to Group 1 | | Group 4 to Group 3 | |
|---|---|---|---|---|---|
| Name | Description | log2 ratio | p values | log2 ratio | p values |

Upregulated genes^{a}

| | | | | | |
|---|---|---|---|---|---|
| Ho-1 | hemeoxygenase 1 | 6.601 | 0.000E+00 | 1.755 | 4.712E−44 |
| Cebpb | CCAAT/enhancer binding protein beta | 2.930 | 4.358E−164 | 1.661 | 1.309E−28 |
| Plpp3 | phospholipid phosphatase 3 | 0.751 | 7.027E−04 | 1.307 | 3.287E−10 |
| Sqstm1 | sequestosome 1 | 1.366 | 1.227E−35 | 1.225 | 2.665E−26 |
| Pik3cb | phosphatidylinositol-4,5-biphosphate | 0.879 | 8.668E−11 | 1.157 | 2.285E−18 |
| | 3-kinase, catalytic subunit beta | | | | |
| Txndc2 | thioredoxin domain containing 2 | 0.917 | 4.981E−02 | 1.153 | 3.690E−03 |
| HspaS | heat shock protein family A | 1.931 | 1.714E−79 | 1.151 | 4.385E−13 |
| | (Hsp70) member 8 | | | | |
| Hsph1 | heat shock protein family H | 4.000 | 0.000E+00 | 1.027 | 2.124E−07 |
| | (Hsp110) member 1 | | | | |
| Ptgds | prostaglandin D2 synthase | −1.115 | 1.478E−04 | 0.954 | 2.334E−03 |
| Nfe2l2 | nuclear factor, erythroid 2-like 2 | 1.898 | 1.063E−43 | 0.923 | 9.336E−11 |
| Plcg2 | phospholipase C, gamma 2 | −1.570 | 3.015E−11 | 0.881 | 4.049E−03 |

Downregulated genes^{b}

| | | | | | |
|---|---|---|---|---|---|
| Nefl | neurofilament light | 0.738 | 1.168E−01 | −1.778 | 5.902E−07 |
| Acsl6 | acyl-CoA synthetase long-chain | −1.225 | 2.073E−06 | −1.582 | 1.693E−05 |
| | family member 6 | | | | |
| Mt2A | metallothionein2A | 2.134 | 5.863E−09 | −1.425 | 9.110E−05 |
| Mt1 | metallothionein 1 | 0.866 | 8.031E−06 | −1.392 | 3.600E−09 |
| Glrx | glutaredoxin | 0.726 | 3.319E−03 | −1.262 | 8.634E−06 |
| Alpl | alkaline phosphatase, | 1.344 | 1.392E−04 | −1.098 | 1.057E−03 |
| | biomineralization associated | | | | |
| Nup43 | nucleoporin 43 | 0.947 | 6.178E−05 | −0.888 | 1.606E−04 |

^{a}Up-regulated significantly (adjusted p-value ≤ 0.01 and/or log2 fold change ≥ 1) in Group 4 compared to Group 3
^{b}Down-regulated significantly (adjusted p-value ≤ 0.01 and/or log2 fold change ≤ −1) in Group 4 compared to Group 3

### Examples 5-1 and 5-2, Comparative Example 2, and Reference Examples 2 and 3: Evaluation of Cell Protection Using Chromogenic Substrate as Indicator (2)

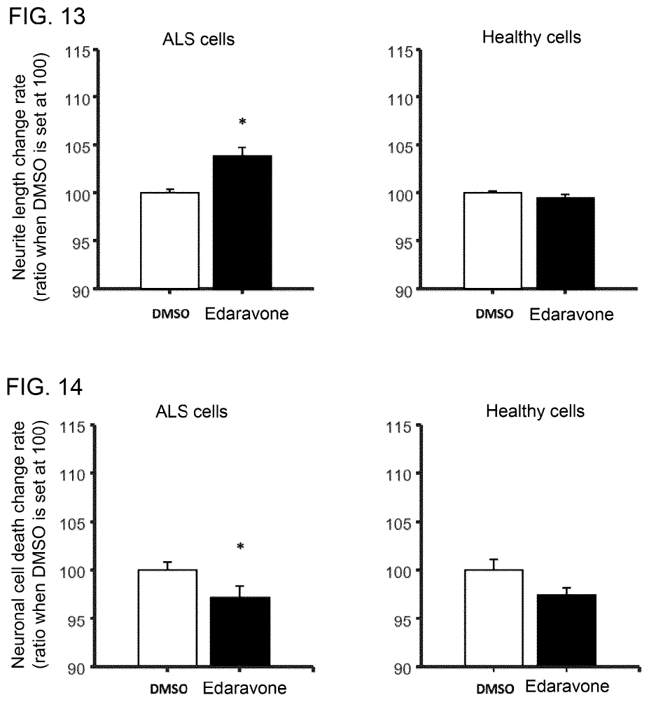

Using the differentiated cell line prepared using the method of Example 1 (indicated as “TDP-43 (−)” in FIG. 12), an Eda-unexposed group (Reference Example 2) and a 96-hour Eda-exposed group (final concentration 100 μmol/L) (Reference Example 3) were each prepared.

Further, using the disease model cells prepared using the method of Example 1 (indicated as “TDP-43 (+)” in FIG. 12), an Eda-unexposed group (Comparative Example 2) and 96-hour Eda-exposed groups (final concentration 50 or 100 μmol/L) (Examples 5-1 and 5-2) were each prepared.

All of these were performed without containing EA.

After that, similar to Example 3-1 and the like, a cell toxicity measurement kit was used to expose the cells in each group according to a product protocol, and an amount of chromogenic substrate produced was measured by absorbance at 450 nm. The results are shown in FIG. 12.

The data shown in FIG. 12 are all expressed as mean±standard error of the mean (SEM, n=4 for each). In FIG. 12, the symbol “##” indicates a statistically significant difference of p<0.01 compared to Comparative Example 2. In FIG. 12, the symbol “**” indicates a statistically significant difference of p<0.05 compared to Comparative Example 2. In FIG. 12, the symbol “$” indicates a statistically significant difference of p<0.05 compared to Reference Example 2 (all determined by Student t-test).

As shown in FIG. 12, in the TDP-43 presence and Eda-containing groups (Examples 5-1 to 5-2), a higher number of viable cells was confirmed compared to the TDP-43 presence and Eda-unexposed group (Comparative Example 2). This indicates that even in the absence of EA, cellular damage occurred in the presence of TDP-43, and that Eda suppressed cellular damage caused by TDP-43.

### Example 6: Preparation of Disease Model Cells (2)

Human induced pluripotent stem (iPS) cells were induced from cells derived from an ALS patient and cells derived from a healthy individual using a conventional method. The cells derived from the ALS patient were used that are known to have an ALS risk mutation as a heterozygous mutation in the TARDBP gene. Each iPS cell was differentiated into a motor neuron (hereinafter, also simply referred to as a “neuron”) using a conventional method. Neurons differentiated from iPS cells induced from cells derived from an ALS patient are simply referred to as “ALS patient-derived neurons,” and neurons differentiated from iPS cells induced from cells derived from a healthy individual are simply referred to as “healthy individual-derived neurons.” For long-term preservation of the cells, when necessary, the neurons were subjected to a freezing treatment 2 to 7 days after the differentiation treatment. Neurons that have been subjected to the freezing treatment are also referred to as “frozen neurons.”

Subsequently, neurons were cultured using a 98-well culture plate (Corning, 3548) under the conditions shown below. Coating of the culture plate was performed in advance before cell seeding. Specifically, a 0.02% Poly-L-Ornithine solution (Sigma-Aldrich, P4957) was added to the wells of the culture plate, and left to stand for 2 hours in a 37° C. 5% CO₂ incubator. After that, the wells were washed, and a 20 mg/mL Laminin solution (Thermo Fisher Scientific, 23017015) was used and it was left to stand for a further 2 hours in a 37° C. 5% CO₂ incubator. After that, the frozen neurons were thawed using a conventional method, and the thawed neurons were seeded into the wells at 20,000 cells/well, and the neurons were cultured in the presence of a culture medium having the following composition.

The culture medium used was a mixture with the following composition.
DMEM/F12 (Thermo Fisher Scientific, 21331-020): 50% v/v
Neurobasal Medium (Thermo Fisher Scientific, 21103-049): 50% v/v
Glutamax Supplement (Thermo Fisher Scientific, 35050061): 1% v/v
Penicillin-Streptomycin (10000 units/mL) (Thermo Fisher Scientific, 15140-148): 0.5% v/v
Component N1 (Elixirgen): 3% v/v
Component A (Elixirgen): 0.1% v/v
Component D4 (Elixirgen): 0.1% v/v
Component P (Elixirgen): 0.05% v/v

### Example 7: Evaluation of Cellular Damage by Live Cell Imaging

A treatment was performed in which the culture medium dissolved with a dimethyl sulfoxide (DMSO) solution of the evaluation compound (edaravone) (final concentration: edaravone 30 μM, DMSO 0.03% v/v) was brought into contact with the neurons after 7 days of cultivation, and occurrence of cellular damage was evaluated over time. Separately, a group without the evaluation compound in the culture medium (a group with the same concentration of DMSO added) was also prepared.

Evaluation of cellular damage was performed by live cell imaging using IncuCyte S3 (Sartorius) and Cytotox (Sartorius, 4846) according to an attached protocol. The neurite length per unit area (unit: mm/mm²) and the number of dead cells (unit: count/image) were analyzed over time and used as evaluation indicators. Smaller values of neurite length and higher numbers of dead cells indicate greater cellular damage.

### Cellular Damage Evaluation (1) Based on Neurite Length

FIG. 13 shows the results of calculating the rate of change in neurite length for each experimental group from the start of the evaluation compound treatment to the time of analysis (24 hours), expressed as a ratio where the DMSO group without the evaluation compound is set to 100. The smaller the value on the vertical axis, the more the neurite length decreases over time, indicating that cellular damage has occurred. The experimental groups in FIG. 13 are as follows.
ALS cells-DMSO: A group using ALS patient-derived neurons, with no edaravone in the culture medium
ALS cells-edaravone: A group using ALS patient-derived neurons, with edaravone in the culture medium
Healthy cells-DMSO: A group using healthy individual-derived neurons, with no edaravone in the culture medium
Healthy cells-edaravone: A group using healthy individual-derived neurons, with edaravone in the culture medium

When the edaravone treatment was applied to both healthy individual-derived neurons and ALS patient-derived neurons, the neurite length in the ALS patient-derived neurons was significantly increased compared to the DMSO-treated group, indicating a protective effect on the neurites. In contrast, no significant change in neurite length was observed in the healthy individual-derived neurons.

### Cellular Damage Evaluation (2) Based on Number of Dead Cells

FIG. 14 shows the results of calculating the rate of change in cell death for each experimental group from the start of the evaluation compound treatment to the time of analysis (24 hours), expressed as a ratio where the DMSO-treated group without the evaluation compound is set to 100. The larger the value on the vertical axis, the greater the number of dead cells over time, indicating that cellular damage has occurred. The experimental groups in FIG. 14 are as follows.
ALS cells-DMSO: A group using ALS patient-derived neurons, with no edaravone in the culture medium
ALS cells-edaravone: A group using ALS patient-derived neurons, with edaravone in the culture medium
Healthy cells-DMSO: A group using healthy individual-derived neurons, with no edaravone in the culture medium
Healthy cells-edaravone: A group using healthy individual-derived neurons, with edaravone in the culture medium

When the edaravone treatment was applied to both healthy individual-derived neurons and ALS patient-derived neurons, the number of dead neurons in the ALS patient-derived neurons was significantly decreased compared to the DMSO-treated group, indicating a protective effect against neuronal cell death. In contrast, no significant change in the number of dead neurons was observed in the healthy individual-derived neurons.

### Example 8: TDP-43 Localization Evaluation

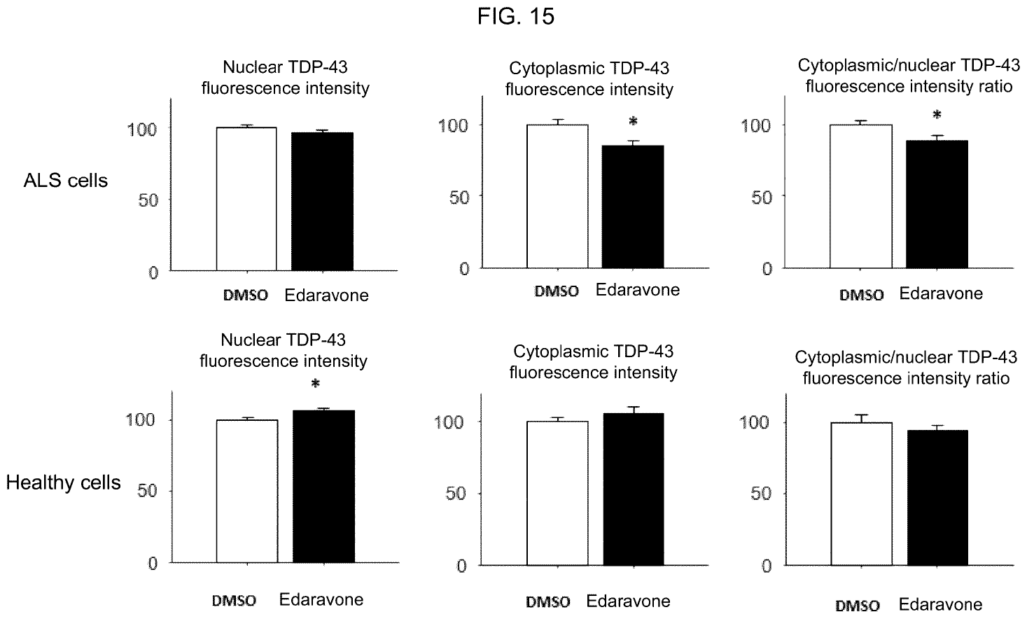

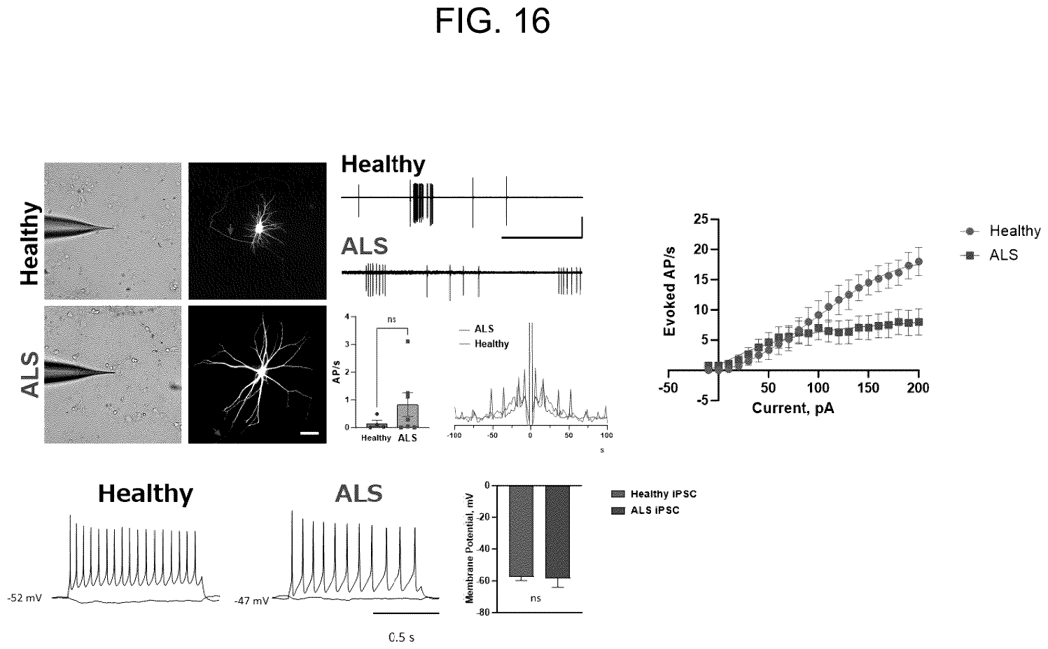

Intracellular localization of TDP-43 polypeptides was evaluated by immunofluorescence staining of neurons. The cultured neurons were washed with phosphate-buffered saline (PBS) and fixed with 4% paraformaldehyde (PFA). After blocking with PBS containing 5% fetal bovine serum (FBS) and 0.1% Triton™-X, a primary antibody dilution containing anti-TDP-43 antibody and anti-β-III tubulin antibody was added and incubated overnight at 4° C. The anti-β-III tubulin antibody was used to visualize the neuronal cell bodies. The next day, after washing with PBS, a secondary antibody dilution conjugated with Alexa dye was added and incubated for 1 hour at room temperature. After further nuclear staining, washing with PBS, and imaging under a fluorescence microscope were performed. The captured images were analyzed using Matlab (Mathworks), and fluorescence intensities of TDP-43 in a nuclear region and in a cytoplasmic region were respectively calculated. The results of the intracellular localization of the TDP-43 polypeptide are shown in FIG. 15. The experimental groups in FIG. 15 are as follows.
ALS cells-DMSO: A group using ALS patient-derived neurons, with no edaravone in the culture medium
ALS cells-edaravone: A group using ALS patient-derived neurons, with edaravone in the culture medium
Healthy cells-DMSO: A group using healthy individual-derived neurons, with no edaravone in the culture medium
Healthy cells-edaravone: A group using healthy individual-derived neurons, with edaravone in the culture medium

Regarding the intracellular localization of TDP-43 polypeptides, the nuclear and cytoplasmic TDP-43 fluorescence intensities and the cytoplasmic/nuclear TDP-43 intensity ratios in ALS cells and healthy cells are shown in FIG. 15. The values are presented as ratios with the DMSO group set to 100. When the edaravone treatment was applied to both healthy individual-derived neurons and ALS patient-derived neurons, the cytoplasmic TDP-43 intensity and the cytoplasmic/nuclear TDP-43 intensity ratio significantly decreased in ALS patient-derived neurons after 24 hours, indicating that the abnormal localization of TDP-43 observed in ALS pathology is suppressed. Further, such an effect of decreasing the cytoplasmic/nuclear TDP-43 intensity ratio was not observed in the healthy individual-derived neurons.

From the above results, it was demonstrated that edaravone, an approved drug for ALS, has the effect of suppressing cellular damage in ALS patient-derived neurons and improving the abnormal intracellular localization of TDP-43. The abnormal localization of TDP-43 is a pathological condition observed in a pathological condition of ALS, which is a heterogeneous disease, and demonstrating an effect in correcting the abnormal localization of TDP-43 indicates the possibility of showing efficacy in ALS patients in general. Further, without being limited to ALS, it can contribute to beneficial effects in the treatment or prevention or the like of diseases related to the abnormal localization of TDP-43. The results indicate that edaravone can be a suitable therapeutic or preventive method for diseases associated with abnormal localization of TDP-43.

### Cell Culture and Multi-Electrode Array

FIG. 16 shows motor neuron activity and response in healthy iPSC-derived motor neurons and ALS iPSC-derived motor neurons (“iPSC” means induced pluripotential stem cell). In FIG. 16, ALS iPSC-derived motor neurons show higher spontaneous activity but lower evoked response compared to heathy iPSC-derive motor neurons.

The induced pluripotent stem cells (iPSCs) were obtained from the National Institute of Neurological Disorders and Stroke (NINDS) and differentiated into cholinergic motor neurons by Elixirgen. The iPSC-derived motor neurons from healthy individuals and ALS patients with TDP43 mutation (A382T) were co-cultured with astrocytes in multi-electrode array (MEA) plates coated with 0.002% poly-ornithine in PBS and 0.8 mg/mL laminin in N1 (AD4P) cell media, which consists of 50% DMEM (Gibco), 50% Neurobasal Medium, 1% penicillin-streptomycin (Gibco), and components N1, A, D4 and P. The iPSC motor neurons and astrocytes were plated at a ratio of 200,000 neurons to 50,000 astrocytes to accelerate neuronal maturation.

After one week of culture, the 50% media was switched to Neurobasal Plus Medium supplemented with B27 Plus, 1% penicillin-streptomycin (Gibco), 200 mM Glutamax, and 200 mM ascorbic acid (Sigma). Multi-electrode plates were recorded weekly for neuronal and network activity using HD-MEA from MaxwellBio. Once network activity plateaued at week 5, the cells were treated with 100 mM edaravone (Sigma) and recordings were taken at 24 hours and 48 hours post-treatment. Edaravone was prepared fresh in 100% DMSO, maintaining a final DMSO concentration of 0.03% in both the vehicle and edaravone treatments.

In addition to recording neuronal activity, electrical stimulation was performed in the presence of edaravone and the vehicle. To test the response of the iPSC neurons to stimulation, thirty selected active electrodes were stimulated with randomization at 10-second intervals, ranging from 100 mV to 500 mV. To measure electrically evoked activity, the Pre/Post stimulus ratio is calculated by dividing the firing frequency during the response to the stimulus by the firing frequency of unstimulated activity over a 1 second period.

First, the neuronal function of induced pluripotent stem cells (iPSCs) derived from healthy individuals and patients with amyotrophic lateral sclerosis (ALS) with TDP-43 mutation (A382T) were evaluated.

The neuronal activity began to develop by week two, and the first network burst was observed by week three and saturated by week five. It was found that the neurons derived from ALS patients with the TDP-43 mutation exhibited significantly different electrophysiological characteristics compared to those from healthy controls. The ALS-derived neurons showed a lower number of active electrodes and an elevated firing rate. Moreover, the bursting activity of the networks differed significantly between the two groups. Neurons from TDP-43 mutation patients not only had a higher bursting frequency.

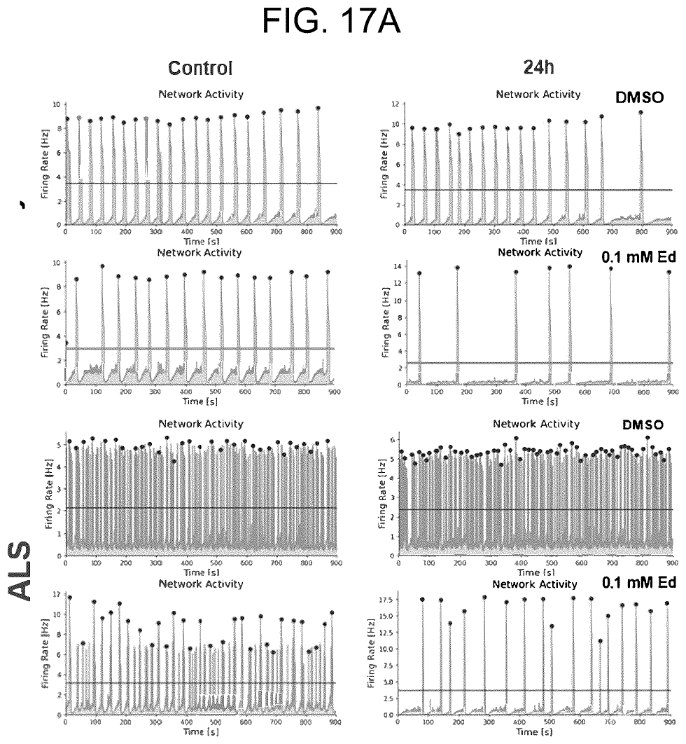

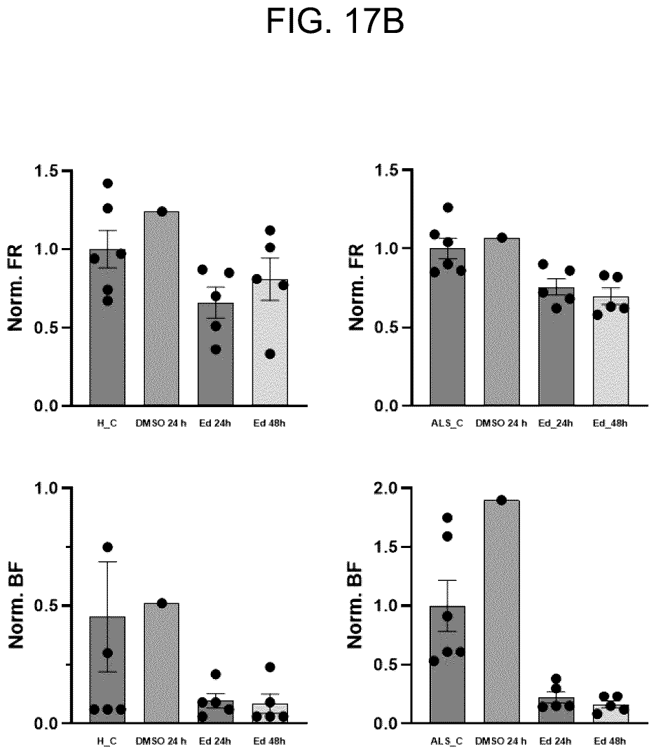

The impact of edaravone treatment on neuronal function in ALS cells with the TDP-43 mutation is evaluated. The iPSC neurons cultured on MEA plates were treated with 100 mM edaravone at week five. FIG. 17A is a representative graph of the network activity shown as a function of firing rate over time for the Healthy and ALS iPSC neurons before and 24 h after the edaravone treatment. FIG. 17B is a normalized bar graph showing the difference in Firing rate and Bursting frequency for healthy and ALS motor neurons before and after the edaravone treatment at 24 and 48 h. This treatment tended to reduce firing rates and network bursts in both healthy and ALS neurons compared to controls.

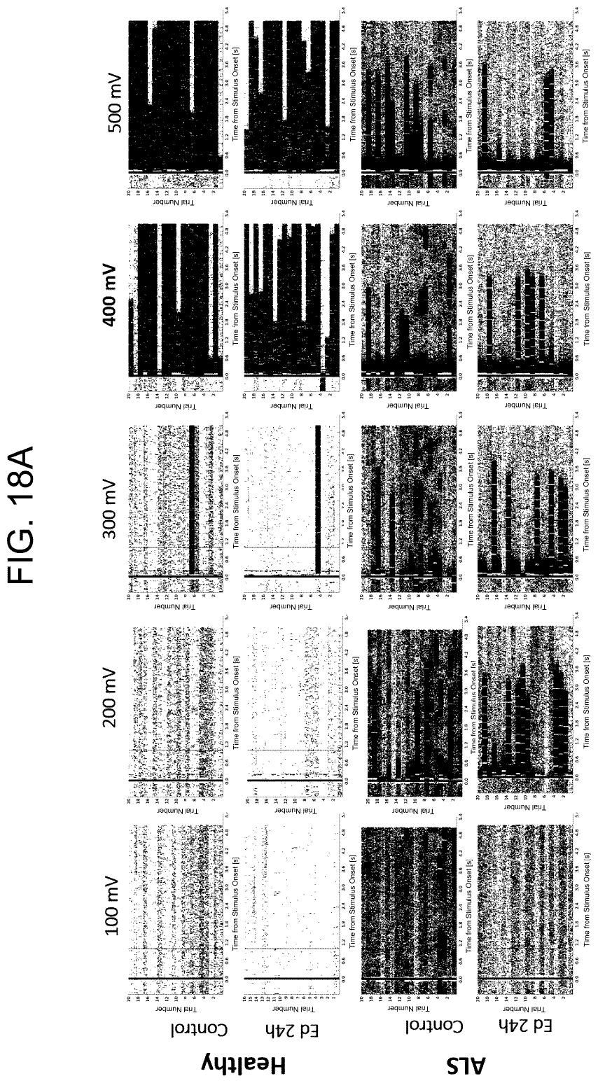

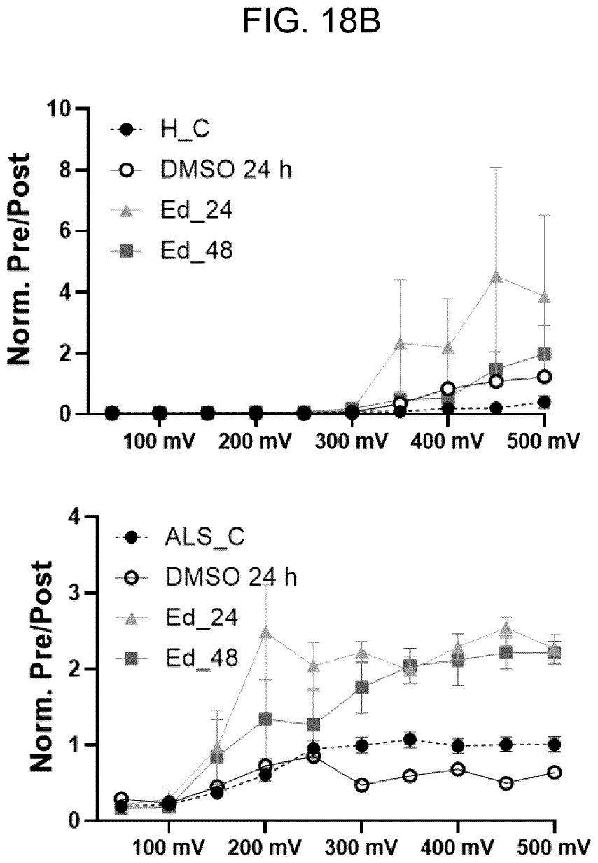

To evaluate elevated resting activity on evoked activity, the iPSC neurons were stimulated with an electrical train pulse for 100 ms. FIG. 18A is panels showing a representative raster plot where the y-axis is several randomized trials, and the x-axis is the time after stimulation. The thirty highly active electrodes were stimulated with a 100 ms pulse train at 10-second intervals with 100 mV increments, ranging from 100 mV to 500 mV. The data is shown for the control and 24 h after 100 mM edaravone treatment for Healthy and ALS neurons with TDP43 mutation.

FIG. 18B shows normalized plots of the mean data points of the Pre/Post stimulus ratio as mean±SEM for both healthy and ALS motor neurons before and after the edaravone treatment at 24 and 48 h.

The ALS neurons TDP-43 mutation demonstrated an early response to the stimulus but quickly reached a plateau compared to neurons from healthy controls. These findings suggest that the TDP-43 mutation impacts the overall dynamics of the neuronal network, leading to an impaired response to stimuli.

The reduction in firing rate and network bursts after edaravone treatment appeared beneficial to neuronal function. Electrically evoked neuronal activity showed improved responses in both healthy and ALS iPSC neurons following edaravone treatment, suggesting that edaravone treatment reduces neuronal noise and improves the fidelity to the stimulus in both healthy and ALS iPSCs and that edaravone ameliorates neuronal responses to stimuli.

ALS is an intractable disease that leads to respiratory failure from initial symptoms such as weakness in hands, movement disorders with fingers and fascicular contraction in upper limbs, through symptoms such as amyotrophia and/or muscular weakness, bulbar paralysis and fascicular contraction in muscles. Neurodegenerative diseases such as ALS have been reported to involve, for example, TDP-43 (TAR DNA-binding protein of 43 kDa) protein. TDP-43 is a protein identified as one of components found in the brain of a patient with frontotemporal lobar degeneration (FTLD) or ALS (see Arai et al., Biochem Biophys Res Commun, 351 (3): 602-11 (2006)). The contents of this publication are incorporated herein by reference. Onset and progression of a neurodegenerative disease such as ALS are thought to be partly due to formation of aggregates of the TDP-43 protein.

It is found that exposure of a target to edaravone causes a change in expression level of a gene product of a specific gene. It is also found that the change in expression level can be a useful indicator, such as a biomarker, in exploring responsiveness to edaravone or possibility of having an applicable disease, or the like.

In one embodiment, the composition is used for causing a change in expression level of a gene product in a target.

In one embodiment, the gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1, and FAIM2.

A method for evaluating responsiveness to edaravone according to one embodiment of the present invention includes evaluating whether or not there is a possibility that a target has responsiveness to edaravone based on a change in expression level of a gene product due to exposure of the target to edaravone.

In one embodiment, the gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1, and FAIM2.

According to an embodiment of the present invention, by exposing a target to edaravone, an expression level of a specific gene product can be changed. As a result, an advantageous effect corresponding to a function of a gene can be achieved, and can be used, for example, in treatment or prevention of a disease.

Further, according to an embodiment of the present invention, an expression change of a specific gene product can be used in screening a substance capable of treating or preventing a disease.

Further, according to an embodiment of the present invention, an expression change of a specific gene product can be used in various assessments and predictions of the presence or absence of a disease and progression of the disease, treatment responsiveness, and the like.

The present specification further includes the following embodiments.

A composition for causing a change in expression level of a gene product in a target according to an embodiment of the present invention includes edaravone. The gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2.

The composition may be used as a pharmaceutical containing edaravone as an active ingredient.

The composition may be used for treating or preventing a neurodegenerative disease.

In the composition, the neurodegenerative disease may be amyotrophic lateral sclerosis (ALS).

In the composition, the target may be preferably a mammal or a sample derived from a mammal, and the mammal is more preferably a human or a rat.

In the composition, the target may be a brain of a mammal or a sample derived from a brain.

The composition may be used to decrease an expression level of a gene product of a first gene in the target having a neurodegenerative disease, and/or used to increase an expression level of a gene product of a second gene in the target having a neurodegenerative disease. The first gene is one or more selected from KAZALD1, SBK1, SCN2A, UBE2L6, ALPL, NTM, PTTG1, ITGB4, HAUS4, DCTD, MT2A, ASF1B, FCSK and MAST1, and the second gene is FAIM2.

In the composition, the target includes one or more selected from neuronal cells (neurons), glial cells, astrocytes and neural stem cells.

In the composition, TDP-43 mutant protein may be present in the target.

A method for evaluating a possibility of having a neurodegenerative disease of a target according to an embodiment of the present invention includes evaluating whether or not there is a possibility that the target has a neurodegenerative disease based on a change in expression level of a gene product in the target. The gene product is a gene product of one or more genes selected from KAZALD1, SBK1, UBE2L6, ALPL, NTM, PTTG1, ITGB4, HAUS4, DCTD, MT2A, ASF1B, FCSK, MAST1 and FAIM2.

In the method, the evaluating may include comparing an expression level of the gene product in the target with an expression level of the gene product in a normal target, and evaluating that the target has a high possibility of having a neurodegenerative disease when there is a change in the expression level of the gene product between the target and the normal target.

In the method, the evaluating that the target has a high possibility of having a neurodegenerative disease when there is a change in the expression level of the gene product between the target and the normal target may be evaluating that the target has a high possibility of having a neurodegenerative disease when the expression level of the gene product of one or more genes selected from KAZALD1, SBK1, UBE2L6, ALPL, NTM, PTTG1, ITGB4, HAUS4, DCTD, MT2A, ASF1B, FCSK, and MAST1 is higher, or when the expression level of the gene product of FAIM2 is lower, compared to the expression level of the gene product in the normal target.

In the method, the gene may be any one of the following (i) to (v): (i) one or more selected from KAZALD1, SBK1, HAUS4, DCTD, FCSK and FAIM2, (ii) one or more selected from KAZALD1, SBK1, DCTD and FCSK, (iii) one or more selected from SBK1, DCTD and FCSK, (iv) one or more selected from DCTD and FCSK, and (v) one or more selected from KAZALD1, SBK1, UBE2L6, NTM, DCTD, ASF1B and FCSK.

In the method, the target may be a brain of a mammal or a sample derived from a brain, and the mammal is preferably a human.

In the method, the target may include one or more selected from neuronal cells (neurons), glial cells, astrocytes and neural stem cells.

In the method, the neurodegenerative disease may be amyotrophic lateral sclerosis (ALS).

A method for evaluating responsiveness of a target to edaravone according to an embodiment of the present invention includes evaluating whether or not there is a possibility that the target has responsiveness to edaravone based on a change in expression level of a gene product due to exposure of the target to edaravone. The gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2, and the target is preferably a target that has or is suspected of having a neurodegenerative disease.

In the method, the evaluating may include comparing an expression level of the gene product in a first target that has or is suspected of having a neurodegenerative disease and has not been exposed to edaravone with an expression level of the gene product in a second target that has or is suspected of having a neurodegenerative disease and has been exposed to edaravone, and evaluating that a target has or is suspected of having a neurodegenerative disease has a high possibility of having responsiveness to edaravone when the expression level of the gene product has changed between the first target and the second target.

In the method, the gene product may be a gene product of one or more genes selected from KAZALD1, SBK1, SCN2A, UBE2L6, ALPL, NTM, PTTG1, ITGB4, HAUS4, DCTD, MT2A, ASF1B, FCSK and MAST1, and the evaluating that a target has or is suspected of having a neurodegenerative disease has a high possibility of having responsiveness to edaravone when the expression level of the gene product has changed between the first target and the second target may be evaluating that a target that has or is suspected of having a neurodegenerative disease has a high possibility of having responsiveness to edaravone when the expression level of the gene product in the second target is lower than the expression level of the gene product in the first target.

In the method, the gene product may be a gene product of FAIM2, and the evaluating that a target has or is suspected of having a neurodegenerative disease has a high possibility of having responsiveness to edaravone when the expression level of the gene product has changed between the first target and the second target may be evaluating that a target that has or is suspected of having a neurodegenerative disease has a high possibility of having responsiveness to edaravone when the expression level of the gene product in the second target is higher than the expression level of the gene product in the first target.

In the method, the evaluating whether or not there is a possibility that the target has responsiveness to edaravone based on a change in expression level of a gene product due to exposure of the target to edaravone may include comparing an expression level of the gene product in a second target that has or is suspected of having a neurodegenerative disease and has been exposed to edaravone with an expression level of the gene product in a normal target; and evaluating that there is a possibility that an effect due to edaravone is high for a target that has or is suspected of having a neurodegenerative disease when there is no change in the expression level of the gene product between the second target and the normal target.

In the method, the evaluating whether or not there is a possibility that the target has responsiveness to edaravone based on a change in expression level of a gene product due to exposure of the target to edaravone may include comparing an expression level of the gene product in a second target that has or is suspected of having a neurodegenerative disease and has been exposed to edaravone with an expression level of the gene product in a normal target; and evaluating that there is a possibility that an effect due to edaravone is low for a target that has or is suspected of having a neurodegenerative disease when there is a change in the expression level of the gene product between the second target and the normal target.

In the method, the neurodegenerative disease may be amyotrophic lateral sclerosis (ALS), and the target is a human or a sample derived from a human.

A method for screening a substance capable of treating or preventing a neurodegenerative disease according to an embodiment of the present invention includes selecting a test substance as a substance capable of treating or preventing a neurodegenerative disease based on a change in expression level of a gene product in a target exposed to the test substance. The gene product is a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2.

In the method, the selecting a test substance as a substance capable of treating or preventing a neurodegenerative disease based on a change in expression level of a gene product in a target exposed to the test substance may include comparing an expression level of the gene product in a target that has or is suspected of having a neurodegenerative disease and has been exposed to the test substance with an expression level of the gene product in a target that has or is suspected of having a neurodegenerative disease and has not been exposed to the test substance and edaravone, and selecting the test substance as a substance capable of treating or preventing a neurodegenerative disease when there is a change in the expression level of the gene product between the target that has been exposed to the test substance and the target that has not been exposed to the test substance and edaravone.

In the method, the selecting a test substance as a substance capable of treating or preventing a neurodegenerative disease based on a change in expression level of a gene product in a target exposed to the test substance may include comparing an expression level of the gene product in a target that has or is suspected of having a neurodegenerative disease and has been exposed to a test substance with an expression level of the gene product in a normal target that has not been exposed to the test substance and edaravone, and selecting the test substance as a substance capable of treating or preventing a neurodegenerative disease when there is no change in the expression level of the gene product between a target that has been exposed to the test substance and a target that has not been exposed to the test substance but has been exposed to edaravone.

In the method, the gene product may be a gene product of one or more genes selected from KAZALD1, SBK1, SCN2A, UBE2L6, ALPL, NTM, PTTG1, ITGB4, HAUS4, DCTD, MT2A, ASF1B, FCSK and MAST1, and the selecting the test substance as a substance capable of treating or preventing a neurodegenerative disease when there is a change in the expression level of the gene product between the target that has been exposed to the test substance and the target that has not been exposed to the test substance and edaravone may be selecting the test substance as a substance capable of treating or preventing a neurodegenerative disease when the expression level of the gene product in a target that has been exposed to the test substance is lower than the expression level of the gene product in a target that has not been exposed to the test substance and edaravone.

In the method, the gene product may be a gene product of FAIM2, and the selecting the test substance as a substance capable of treating or preventing a neurodegenerative disease when there is a change in the expression level of the gene product between the target that has been exposed to the test substance and the target that has not been exposed to the test substance and edaravone may be selecting the test substance as a substance capable of treating or preventing a neurodegenerative disease when the expression level of the gene product in a target that has been exposed to the test substance is higher than the expression level of the gene product in a target that has not been exposed to the test substance and edaravone.

In the method, the selecting a test substance as a substance capable of treating or preventing a neurodegenerative disease based on a change in expression level of a gene product in a target exposed to the test substance may include comparing an expression level of the gene product in a target that has or is suspected of having a neurodegenerative disease and has been exposed to a test substance with an expression level of the gene product in a target that has or is suspected of having a neurodegenerative disease and has not been exposed to a test substance but has been exposed to edaravone, and evaluating that the test substance has a high possibility of being a substance capable of treating or preventing a neurodegenerative disease when there is no change in the expression level of the gene product between a target that has been exposed to the test substance and a target that has not been exposed to the test substance but has been exposed to edaravone.

In the method, the selecting a test substance as a substance capable of treating or preventing a neurodegenerative disease based on a change in expression level of a gene product in a target exposed to the test substance may include comparing an expression level of the gene product in a target that has or is suspected of having a neurodegenerative disease and has been exposed to a test substance with an expression level of the gene product in a target that has or is suspected of having a neurodegenerative disease and has not been exposed to a test substance but has been exposed to edaravone, and evaluating that the test substance has a low possibility of being a substance capable of treating or preventing a neurodegenerative disease when there is a change in the expression level of the gene product between a target that has been exposed to the test substance and a target that has not been exposed to the test substance but has been exposed to edaravone.

In the method, the neurodegenerative disease may be amyotrophic lateral sclerosis (ALS).

In the method, the target may be a human or a sample derived from a human.

A biomarker for diagnosing a neurodegenerative disease or for diagnosing responsiveness to edaravone according to an embodiment of the present invention includes a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2, preferably one or more genes selected from KAZALD1, SBK1, UBE2L6, NTM, DCTD, ASF1B and FCSK.

A detection kit according to an embodiment of the present invention includes a detection reagent capable of specifically detecting gene products of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2, for detecting one or more of the gene products as biomarkers for diagnosing a neurodegenerative disease or for diagnosing responsiveness to edaravone.

A method for treating a neurodegenerative disease according to an embodiment of the present invention includes administering the composition to a patient with a neurodegenerative disease.

An embodiment of the present invention is use of a substance for causing a change in expression level of a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1, and FAIM2, for manufacturing a composition for prevention or treatment of a neurodegenerative disease.

A method for suppressing occurrence or progression of cellular damage according to an embodiment of the present invention includes causing a change in expression level of a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1 and FAIM2 in cells.

An embodiment of the present invention is edaravone or an edaravone-containing pharmaceutical composition for use in treatment or prevention of a neurode generative disease, such as ALS, by causing a change in expression level of a gene product of one or more genes selected from KAZALD1, SBK1, SCN2A, UBE2L6, ALPL, NTM, PTTG1, ITGB4, HAUS4, DCTD, MT2A, ASF1B, FCSK, MAST1, and FAIM2.

In the composition, method, biomarker, detection kit, or use, the gene may be one, any combination of two or more, or all selected from KAZALD1, SBK1, UBE2L6, ALPL, NTM, PTTG1, ITGB4, HAUS4, DCTD, MT2A, ASF1B, FCSK, MAST1 and FAIM2, preferably, the gene is one, any combination of two or more, or all selected from KAZALD1, SBK1, UBE2L6, NTM, HAUS4, DCTD, ASF1B, FCSK and FAIM2, more preferably, the gene is one, any combination of two or more, or all selected from KAZALD1, SBK1, DCTD and FCSK, even more preferably, the gene is one, any combination of two, or all selected from SBK1, DCTD and FCSK, and most preferably, the gene is one or two selected from DCTD and FCSK.

A method for treating ALS associated with mutant TDP-43 protein according to an embodiment of the present invention includes administering edaravone to a patient in need thereof. The patient has the mutant TDP-43 protein.

A method for treating a disease associated with abnormal intracellular localization of TDP-43 protein according to an embodiment of the present invention includes administering edaravone to a subject in need thereof. The subject has the mutant TDP-43 protein.

A method for suppressing a symptom associated with mutation of TDP-43 protein or improving motor function impairment associated with mutation of TDP-43 protein according to an embodiment of the present invention includes administering edaravone to a patient in need thereof. The patient has the mutant TDP-43 protein.

Obviously, numerous modifications and variations of the present invention are possible in light of the above teachings. It is therefore to be understood that within the scope of the appended claims, the invention may be practiced otherwise than as specifically described herein.

1. A method for evaluating responsiveness of a target to edaravone, comprising:
administering a composition comprising edaravone to a target in need thereof such that the edaravone causes a change in expression level of a gene product in the target; and
evaluating whether the target has responsiveness to edaravone based on the change in expression level of the gene product due to exposure of the target to the edaravone,
wherein the gene product is a gene product of at least one gene selected from the group consisting of KAZALD1, SBK1, SCN2A, UBE2L6, ALPL, NTM, PTTG1, ITGB4, HAUS4, DCTD, MT2A, ASF1B, FCSK, MAST1 and FAIM2.

2. The method of claim 1, wherein the target is a mammal.

3. The method of claim 1, wherein the target is a sample derived from a mammal.

4. The method of claim 1, further comprising:
treating the target for a neurodegenerative disease,
wherein the treating includes administering a pharmaceutical composition comprising edaravone to the target.

5. The method of claim 1, further comprising:
preventing the target from a neurodegenerative disease,
wherein the preventing includes administering a pharmaceutical composition comprising edaravone to the target.

6. The method of claim 4, wherein the neurodegenerative disease is amyotrophic lateral sclerosis.

7. The method of claim 5, wherein the neurodegenerative disease is amyotrophic lateral sclerosis.

8. The method of claim 2, further comprising:
treating the target for a neurodegenerative disease,
wherein the treating includes administering a pharmaceutical composition comprising edaravone to the target.

9. The method of claim 2, further comprising:
preventing the target from a neurodegenerative disease,
wherein the preventing includes administering a pharmaceutical composition comprising edaravone to the target.

10. The method of claim 8, wherein the neurodegenerative disease is amyotrophic lateral sclerosis.

11. The method of claim 9, wherein the neurodegenerative disease is amyotrophic lateral sclerosis.

12. The method of claim 3, further comprising:
treating the target for a neurodegenerative disease,
wherein the treating includes administering a pharmaceutical composition comprising edaravone to the target.

13. The method of claim 3, further comprising:
preventing the target from a neurodegenerative disease,
wherein the preventing includes administering a pharmaceutical composition comprising edaravone to the target.

14. The method of claim 12, wherein the neurodegenerative disease is amyotrophic lateral sclerosis.

15. The method of claim 13, wherein the neurodegenerative disease is amyotrophic lateral sclerosis.

16. A method for evaluating a target for a neurodegenerative disease, comprising:
administering a composition comprising edaravone to a target in need thereof; and
evaluating whether the target has a neurodegenerative disease based on a change in expression level of a gene product in the target,
wherein the gene product is a gene product of at least one gene selected from the group consisting of KAZALD1, SBK1, UBE2L6, NTM, DCTD, ASF1B and FCSK.

17. A method for screening a substance for treating or preventing a neurodegenerative disease, comprising:
administering a composition comprising edaravone to a target in need thereof such that the edaravone causes a change in expression level of a gene product in the target; and
selecting a test substance that treats or prevents a neurodegenerative disease based on the change in expression level of the gene product due to exposure of the target to the test substance,
wherein the gene product is a gene product of at least one gene selected from the group consisting of KAZALD1, SBK1, SCN2A, UBE2L6, ALPL, NTM, PTTG1, ITGB4, HAUS4, DCTD, MT2A, ASF1B, FCSK, MAST1 and FAIM2.

18. A biomarker for diagnosing a neurodegenerative disease, comprising:
a gene product comprising at least one gene selected from the group consisting of KAZALD1, SBK1, UBE2L6, NTM, DCTD, ASF1B and FCSK.

19. A biomarker for diagnosing edaravone responsiveness, comprising:
a gene product comprising at least one gene selected from the group consisting of KAZALD1, SBK1, SCN2A, UBE2L6, ALPL, NTM, PTTG1, ITGB4, HAUS4, DCTD, MT2A, ASF1B, FCSK, MAST1 and FAIM2.
